(19) United States
(12) Patent Application Publication
(10) Pub. No.: US 2024/0041741 A1
(43) Pub. Date: Feb. 8, 2024

(54) COSMETIC COMPOSITION OBTAINED BY MIXING TWO SILANE BLENDS

(71) Applicant: Henkel AG & Co. KGaA, Duesseldorf (DE)

(72) Inventors: Phillip JAISER, Langenfeld (DE); Torsten LECHNER, Langenfeld (DE); Gabriele WESER, Essen (DE); Marc NOWOTTNY, Moenchengladbach (DE); Juergen SCHOEPGENS, Schwalmtal (DE); Andreas WALTER, Ratingen (JP); Ulrike SCHUMACHER, (US)

(73) Assignee: Henkel AG & Co. KGaA, Duesseldorf (DE)

(21) Appl. No.: 18/041,931

(22) PCT Filed: Jun. 21, 2021
(86) PCT No.: PCT/EP2021/066760

§ 371 Date: Feb. 16, 2023

(30) Foreign Application Priority Data
Aug. 17, 2020 (DE) 102020210426.3

(51) Int. Cl. A61K 8/58; A61K 8/19; A61Q 5/06

(52) U.S. Cl. CPC A61K 8/585; A61K 8/19; A61Q 5/065; A61K 2800/882; A61K 2800/432

(57) ABSTRACT

The subject of the present application is a cosmetic composition for the treatment of keratinous material, in particular human hair, obtained by mixing a first agent (A) with a second agent (B), wherein
the first agent (A) is obtained by reacting one or more organic C₁-C₆ alkoxysilanes with an amount of water equal to the molar amount of water determined according to equation (G-1),

X=[(n_{I}(Alkoxysilane)×n_{II}(Alkoxy groups)]/n(H₂O) (G-1)

Where the terms used in equation (G-1) are defined.
the second agent (B) is obtained by reacting one or more organic C₁-C₆ alkoxysilanes with an amount of water equal to the molar amount of water determined according to equation (G-2),

Y=[(nₐ(Alkoxysilane)×n_{b}(Alkoxy groups)]/m(H₂O) (G-2)

Where the terms used in equation (G-2) are defined.

### CROSS-REFERENCE TO RELATED PATENT APPLICATIONS

This application is a U.S. National-Stage entry under 35 U.S.C. § 371 based on International Application No. PCT/EP2021/066760, filed Jul. 21, 2021, which was published under PCT Article 21(2) and which claims priority to German Application No. 10 2020 210 426.3, filed Aug. 17, 2020, which are all hereby incorporated in their entirety by reference.

### TECHNICAL FIELD

The present application relates to a cosmetic composition obtained by mixing the two agents (A) and (B). Agents (A) and (B) are two silane blends, each obtained by reacting one or more organic C₁-C₆ alkoxysilanes with a given amount of water.

A second object of the present disclosure is a multi-component packaging unit (kit-of-parts) for coloring keratinous material, which comprises, separately packaged in two packaging units, the cosmetic preparation described above and further an agent (C), wherein the agent (C) contains at least one coloring compound.

A third object is a process for coloring keratinous material, in which the cosmetic composition and agent (C) described above are applied to the keratinous material.

### BACKGROUND

The change in shape and color of keratin fibers, especially hair, is a key area of modern cosmetics. To change the hair color, the expert knows various coloring systems depending on coloring requirements. Oxidation dyes are usually used for permanent, intensive dyeing with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing obtained with direct dyes have a shorter shelf life and quicker wash ability. Dyes with direct colorings usually remain on the hair for a period of between 5 and about 20 washes.

The use of color pigments is known for short-term color changes on the hair and/or skin. Color pigments are understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

EP 2168633 B1 deals with the task of producing long-lasting hair colorations using pigments. The document teaches that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these hydrolysis or condensation products are applied to keratinous material, a film or coating is formed on the keratinous material, which completely envelops the keratinous material and, in this way, strongly influences the properties of the keratinous material. Areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers, and surprisingly wash-resistant colorations result.

### BRIEF SUMMARY

Cosmetic compositions, kits-of-parts, and methods for dying keratinous material are provided. In an exemplary embodiment, a cosmetic composition includes a first agent (A) and a second agent (B) that are mixed together. The first agent (A) is obtained by reacting one or more organic C₁-C₆ alkoxysilanes with an amount of water determined according to equation (G-1),

X=[(n_{I}(Alkoxysilane)×n_{II}(Alkoxy groups)]/n(H₂O) (G-1)

where n(H₂O) is the amount of water used in the agent (A) expressed in moles, m is the amount of the organic C₁-C₆ alkoxysilanes used in the agent (A) expressed in moles, n_{I} is the number of C₁-C₆ alkoxy groups per the organic C₁-C₆ alkoxysilane used in the agent (A), and X is a number from about 3.0 to about 100. The second agent (B) is obtained by reacting one or more organic C₁-C₆ alkoxysilanes with an amount of water, wherein the amount of water is determined according to equation (G-2),

Y=[(nₐ(Alkoxysilane)×n_{b}(Alkoxy groups)]/m(H₂O) (G-2)

where m(H₂O) is the amount of water used in the agent (B) expressed in moles, nₐ is the amount of the organic C₁-C₆ alkoxysilanes used in the agent (B) expressed in moles, n_{b} is the number of C₁-C₆ alkoxy groups per the organic C₁-C₆ alkoxysilane used in the agent (B), and Y is a number from about 0.1 to about 2.9.

A multicomponent packaging unit, or a kit-of-parts, is provided in another embodiment. The kit-of-parts includes a first packaging unit that includes a cosmetic composition, where the cosmetic composition includes a first agent (A) and a second agent (B) that are mixed together. The first agent (A) is obtained by reacting one or more organic C₁-C₆ alkoxysilanes with an amount of water determined according to equation (G-1),

X=[(n_{I}(Alkoxysilane)×n_{II}(Alkoxy groups)]/n(H₂O) (G-1)

where n(H₂O) is the amount of water used in the agent (A) expressed in moles, n_{I} is the amount of the organic C₁-C₆ alkoxysilanes used in the agent (A) expressed in moles, n_{I} is the number of C₁-C₆ alkoxy groups per the organic C₁-C₆ alkoxysilane used in the agent (A), and X is a number from about 3.0 to about 100. The second agent (B) is obtained by reacting one or more organic C₁-C₆ alkoxysilanes with an amount of water, wherein the amount of water is determined according to equation (G-2),

Y=[(nₐ(Alkoxysilane)×n_{b}(Alkoxy groups)]/m(H₂O) (G-2)

where m(H₂O) is the amount of water used in the agent (B) expressed in moles, nₐ is the amount of the organic C₁-C₆ alkoxysilanes used in the agent (B) expressed in moles, n_{b} is the number of C₁-C₆ alkoxy groups per the organic C₁-C₆ alkoxysilane used in the agent (B), and Y is a number from about 0.1 to about 2.9. The kit-of-parts also includes a second packaging unit that contains an agent (C), where the agent (C) includes at least one pigment and/or direct dye. The kit-of-parts optionally further includes a third packaging unit containing an agent (D), where the agent (D) includes a film-forming polymer.

A method of dying keratinous material in provided in yet another embodiment. The method includes (1) providing a cosmetic composition, where the cosmetic composition includes a first agent (A) and a second agent (B) that are mixed together. The first agent (A) is obtained by reacting one or more organic C₁-C₆ alkoxysilanes with an amount of water determined according to equation (G-1),

X=[(n_{I}(Alkoxysilane)×n_{II}(Alkoxy groups)]/n(H₂O) (G-1)

where n(H₂O) is the amount of water used in the agent (A) expressed in moles, m is the amount of the organic C₁-C₆ alkoxysilanes used in the agent (A) expressed in moles, n_{I} is the number of C₁-C₆ alkoxy groups per the organic C₁-C₆ alkoxysilane used in the agent (A), and X is a number from about 3.0 to about 100. The second agent (B) is obtained by reacting one or more organic C₁-C₆ alkoxysilanes with an amount of water, wherein the amount of water is determined according to equation (G-2),

Y=[(nₐ(Alkoxysilane)×n_{b}(Alkoxy groups)]/m(H₂O) (G-2)

where m(H₂O) is the amount of water used in the agent (B) expressed in moles, nₐ is the amount of the organic C₁-C₆ alkoxysilanes used in the agent (B) expressed in moles, n_{b} is the number of C₁-C₆ alkoxy groups per the organic C₁-C₆ alkoxysilane used in the agent (B), and Y is a number from about 0.1 to about 2.9. The kit-of-parts also includes a second packaging unit that contains an agent (C), where the agent (C) includes at least one pigment and/or direct dye. The kit-of-parts optionally further includes a third packaging unit containing an agent (D), where the agent (D) includes a film-forming polymer. The method further includes (2) providing an agent (C) that includes a pigment and/or a direct dye, and (3) mixing the cosmetic composition from step (1) with the agent (C) to produce a ready-to-use colorant. In step (4) of the method, the ready-to-use colorant is applied onto the keratinous material, and in step (5) the keratinous material is exposed to the ready-to-use colorant. In step (6), the ready-to-use colorant is rinsed out of the keratinous material.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The great advantage of the alkoxy silane-based dyeing principle is that the high reactivity of this class of compounds enables extremely fast coating. This means that extremely good coloring results can be achieved after short application periods of just a few minutes. In addition to these advantages, however, the high reactivity of alkoxy silanes also has some disadvantages. Thus, even minor changes in production and application conditions, such as changes in humidity and/or temperature, can lead to sharp fluctuations in product performance. Most importantly, the work leading to this disclosure has shown that the alkoxy silanes are extremely sensitive to the conditions encountered in the manufacture of the keratin treatment agents.

Further analytical studies have shown that complex hydrolysis and condensation reactions take place during the preparation of various silane or siloxane mixtures and blends, leading to oligomeric products of different molecular size and degree of crosslinking, depending on the reaction conditions selected. In this context, it has been found that the molecular weight of these silane oligomers can have a major influence on the subsequent product properties. If wrong conditions are selected during production, this can lead to the formation of silane condensates that are too large or too small, which negatively affects the subsequent product performance, especially the subsequent dyeing capacity on the keratin material.

It was the task of the present application to find optimized agents and methods for the treatment of human hair. The mixtures of alkoxy siloxanes used in these agents or processes were to be prepared in a targeted manner so that the optimum application properties could be achieved in a subsequent application. Agents prepared in this way should have an optimal degree of crosslinking or siloxane oligomers with optimal molecular weight distribution, which should result in improved dyeing performance. In this way, the agents, when applied in a dyeing process, should result in dyeing with higher color intensity and improved fastness properties, especially with improved wash fastness and improved rub fastness. Furthermore, agents prepared in this way should be particularly stable in storage, and the subsequent colorimetric potential of the agents should not depend on the storage time.

Surprisingly, it has now been found that the task can be excellently solved if cosmetic compositions are applied to the keratin material, which are prepared by mixing two agents (A) and (B) before application. Both agents (A) and (B) represent silane blends obtained by reacting C₁-C₆ alkoxy silanes with a given amount of water in each case. In agent (A), the C₁-C₆ alkoxy silanes are reacted with a smaller amount of water, whereas in agent (B), a higher amount of water is reacted with the C₁-C₆ alkoxy silanes.

A first object of the present disclosure is a cosmetic composition for treating keratinous material, in particular human hair, obtained by mixing a first agent (A) with a second agent (B), wherein the first agent (A) is obtained by reacting one or more organic C₁-C₆ alkoxysilanes with an amount of water equal to the molar amount of water determined according to equation (G-1),

X=[(n_{I}(Alkoxysilane)×n_{II}(Alkoxy groups)]/n(H₂O) (G-1)

where
n(H₂O) is the molar amount of water used in the agent (A),
n_{I} is the molar amount of the organic C₁-C₆ alkoxysilanes used in the agent (A),
n_{II} is the number of C₁-C₆ alkoxy groups per C₁-C₆ organic alkoxysilane used in the agent (A), and
X is a number from about 3.0 to about 100, and
the second agent (B) is obtained by reacting one or more organic C₁-C₆ alkoxysilanes with an amount of water equal to the molar amount of water determined according to equation (G-2),

Y=[(nₐ(Alkoxysilane)×n_{b}(Alkoxy groups)]/m(H₂O) (G-2)

where
m(H₂O) is the molar amount of water used in the agent (B),
nₐ is the molar amount of the organic C₁-C₆ alkoxysilanes used in the agent (B),
n_{b} is the number of C₁-C₆ alkoxy groups per C₁-C₆ organic alkoxysilane used in the agent (B), and
Y is a number from about 0.1 to about 2.9.

It has been found that the initial separate preparation of the two agents (A) and (B) and the subsequent mixing of these two agents results in a silane mixture that is particularly stable. Although the mixture of partially reacted or oligomerized C₁-C₆ alkoxy silanes prepared in this way is still reactive, it is surprisingly little affected by external parameters occurring during storage, such as temperature, humidity, and storage period. Even when applied to the hair at a later stage, the agents produced in this way form particularly stable and reproducible films or coatings and show greater independence from the duration of application.

Furthermore, it has been shown that the cosmetic compositions prepared by mixing the two agents (A) and (B), when used in a dyeing process, resulted in very intense and uniform dyeing with particularly good hiding power, rub fastness and wash fastness.

### Keratinous Material

Keratinous material includes hair, skin, nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

### Agent for the Treatment of Keratinous Material

Agents for treating keratinous material are understood to mean, for example, techniques for coloring the keratinous material, techniques for reshaping or shaping keratinous material, in particular keratinous fibers, or also techniques for conditioning or caring for the keratinous material. The agents prepared via the process according to the present disclosure show particularly good suitability for coloring keratinous material, for coloring keratinous fibers, which are especially preferably human hair.

The term “agent for coloring” is used in the context of the present disclosure for coloring of the keratin material, of the hair, caused using coloring compounds, such as pigments, mica, thermochromic and photochromic dyes, direct dyes and/or oxidation dyes. In this staining process, the colorant compounds are deposited in a particularly homogeneous and smooth film on the surface of the keratin material or diffuse into the keratin fiber. The film is formed in situ by oligomerization or condensation of the organic silicon compound(s), with the colorant compound(s) interacting with and being incorporated into, surrounded by this film, or coating.

### Agent (A)

The agent (A) is obtained by reacting one or more organic C₁-C₆ alkoxysilanes with a certain amount of water, the amount of water used (calculated in moles of water) being given by formula (G-1). The agent (A) may alternatively be referred to as a silane blend and comprises one or more silanes that condense upon reaction with the water to form siloxanes and that have a specific molecular weight distribution and oligomeric structure.

Organic C₁-C₆ Alkoxy Silanes in the Agent (A)

The organic C₁-C₆ alkoxy silane(s) are organic, non-polymeric silicon compounds, preferably selected from the group of silanes containing one, two or three silicon atoms.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds of the present disclosure are preferably compounds containing one to three silicon atoms. Organic silicon compounds preferably contain one or two silicon atoms.

According to IUPAC rules, the term silane chemical compounds based on a silicon skeleton and hydrogen. In organic silanes, the hydrogen atoms are completely or partially replaced by organic groups such as (substituted) alkyl groups and/or alkoxy groups.

A typical feature of the C₁-C₆ alkoxy silanes of the present disclosure is that at least one C₁-C₆ alkoxy group is directly bonded to a silicon atom. The C₁-C₆ alkoxy silanes according to the present disclosure thus comprise at least one structural unit R′R″R′″Si—O—(C₁-C₆-Alkyl) where the radicals R′, R″ and R′″ represent the three-remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolysable groups per molecule. If the hydrolysable C₁-C₆ alkoxy group is an ethoxy group, the organic silicon compound preferably contains a structural unit R′R″R′″ Si—O—CH2-CH3. The radicals R′, R″ and R′″ again represent the three remaining free valences of the silicon atom.

In a very particularly preferred embodiment, the first agent (A) is obtained by reacting one or more organic C₁-C₆ alkoxysilanes of formula (I) and/or (II) and/or (IV) with a certain amount of water.

In a very particularly preferred embodiment, a cosmetic composition according to the present disclosure is exemplified in that the first agent (A) is obtained by reacting one or more organic C₁-C₆ alkoxysilanes of formula (I) and/or (II) and/or (IV),

R₁R₂N-L-Si(OR₃)ₐ(R₄)_{b} (I)

where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a, stands for an integer from 1 to 3, and
b stands for the integer 3-a, and

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),

-(A″″)-Si(R₆″)_{d}″(OR₅″)_{c}″ (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h radicals are different from 0,

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. Most preferably, the radicals R₁ and R₂ both represent a hydrogen atom.

In the middle part of the organic silicon compound is the structural unit or the linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each—L grouping may form—two bonds.

Preferably -L- stands for a linear, bivalent C₁-C₂₀ alkylene group. Further preferably -L- stands for a linear bivalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—).

The organic silicon compounds of formula (I)

R₁R₂N-L-Si(OR₃)ₐ(R₄)_{b} (I),

each carry the silicon-containing group —Si(OR₃)ₐ(R₄)_{b}.

In the terminal structural unit —Si(OR₃)ₐ(R₄)_{b} radicals R₃ and R₄ independently represent a C₁-C₆ alkyl group, and particularly preferably R₃ and R₄ independently represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

It has been found to be particularly preferred if, for the preparation of the first agent (A), at least one organic C₁-C₆ alkoxy silane of the formula (I) has been reacted with water, in which the radicals R₃, R₄ independently of one another represent a methyl group or an ethyl group.

Furthermore, particularly satisfactory results were obtained when, for the preparation of the first agent (A), at least one organic C₁-C₆ alkoxy silane of the formula (I) was reacted with water, in which the radical a represents the number 3. In this case the radial b stands for the number 0.

In another preferred embodiment, a cosmetic composition according to the present disclosure, is exemplified in that the first agent (A) is obtained by reacting one or more organic C₁-C₆ alkoxysilanes of formula (I) with water,

where
R₃, R₄ independently of one another represent a methyl group or an ethyl group and
a stands for the number 3 and
b stands for the number 0.

Organic silicon compounds of the formula (I) which are particularly suitable for solving the problem according to the present disclosure are

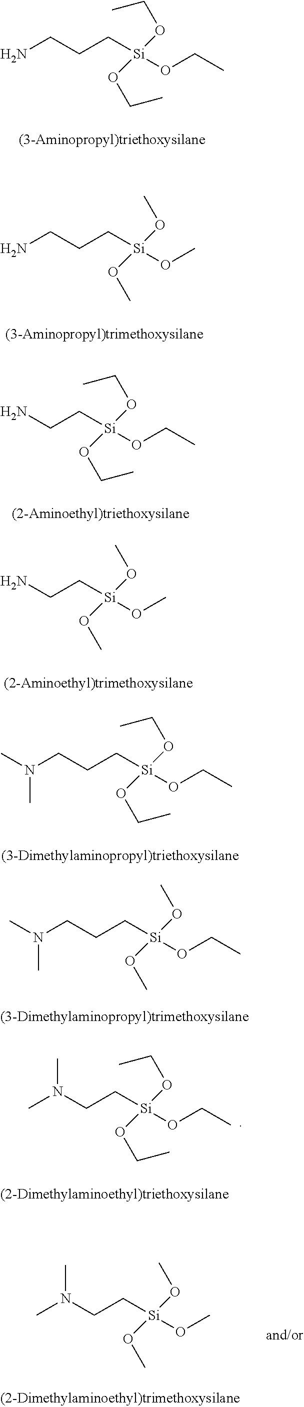

The organic silicon compound of formula (I) is commercially available. (3-aminopropyl)trimethoxysilane, for example, can be purchased from Sigma-Aldrich®. Also (3-aminopropyl)triethoxysilane is commercially available from Sigma-Aldrich®.

In another preferred embodiment, a cosmetic composition according to the present disclosure is exemplified in that the first agent (A) is obtained by reacting one or more organic C₁-C₆ alkoxysilanes of formula (II) with a certain amount of water,

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II).

The organosilicon compounds of formula (II) according to the present disclosure each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) according to the present disclosure contains at least one grouping from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}′, the radicals R5, R5′, R5″ independently represent a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeing with the best wash fastness values could be obtained if the residues c and c′ both stand for the number 3. In this case d and d′ both stand for the number 0.

In another preferred embodiment, a cosmetic composition according to the present disclosure is exemplified in that the first agent (A) is obtained by reacting one or more organic C₁-C₆ alkoxysilanes of formula (II) with water,

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and —[NR₇-(A′)]_{f}- and —[O-(A″)]_{g}- and —[NR₈-(A′″)]ₕ- are in the middle part of the organic silicon compound of formula (II).

In this context, the presence of certain groupings has proven to be particularly advantageous in terms of achieving washfast dyeing results. Particularly satisfactory results could be obtained if at least two of the residues e, f, g, and h stand for the number 1. Especially preferred e and f both stand for the number 1. Furthermore, g and h both stand for the number 0.

If e and f both stand for the number 1 and g and h both stand for the number 0, the organic silicon compound according to the present disclosure corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A″″ independently represent a linear or divalent, bivalent C₁-C₂₀ alkylene group. Preferably the radicals A, A′, A″, A′″ and A″″ independently of one another represent a linear, bivalent C₁-C₂₀ alkylene group. Further preferably the radicals A, A′, A″, A′″ and A″″ independently represent a linear bivalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A′″ and A″″ represent a propylene group (—CH₂—CH₂—CH₂—).

If the radical f represents the number 1, then the organic silicon compound of formula (II) according to the present disclosure contains a structural grouping —[NR₇-(A′)]-. If the radical f represents the number 1, then the organic silicon compound of formula (II) according to the present disclosure contains a structural grouping —[NR₈-(A′″)]-.

Wherein radicals R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)-Si(R₆″)_{d}″(OR₅′″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound according to the present disclosure contains the grouping [NR₇-(A′)] but not the grouping —[NR₈-(A′″)]. If the radical R7 now stands for a grouping of the formula (III), the pretreatment agent (a) contains an organic silicon compound with 3 reactive silane groups.

In another preferred embodiment, a cosmetic composition according to the present disclosure is exemplified in that the agent (A) is obtained by reacting one or more organic C₁-C₆ alkoxysilanes of formula (II) with water,

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group, and
R7 represents a hydrogen atom, a methyl group, a 2-hydroxyethyl group, a 2-alkenyl group, a 2-aminoethyl group or a group of formula (III).

In a further preferred embodiment, a cosmetic composition according to the present disclosure is exemplified in that the agent (A) is obtained by reacting one or more organic C₁-C₆ alkoxysilanes of formula (II) with water, wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem according to the present disclosure are

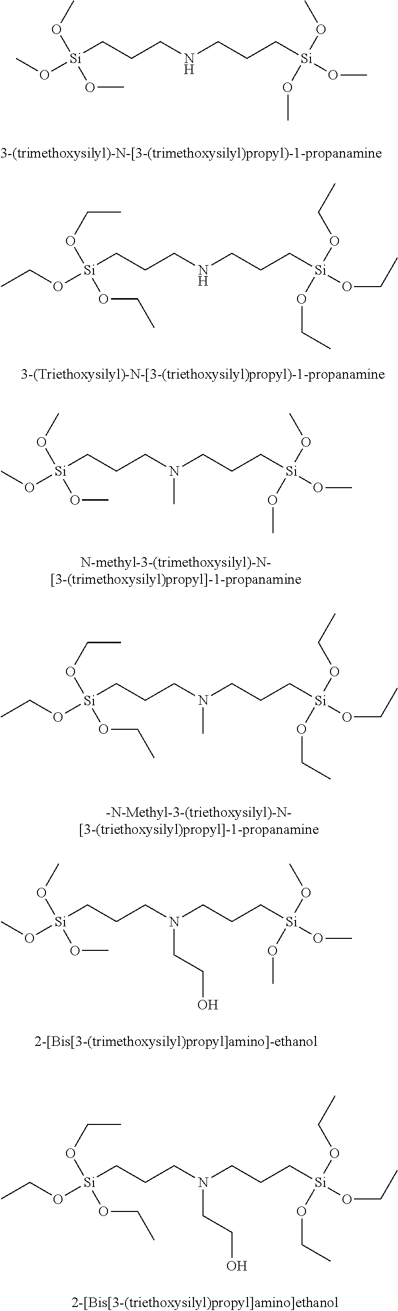

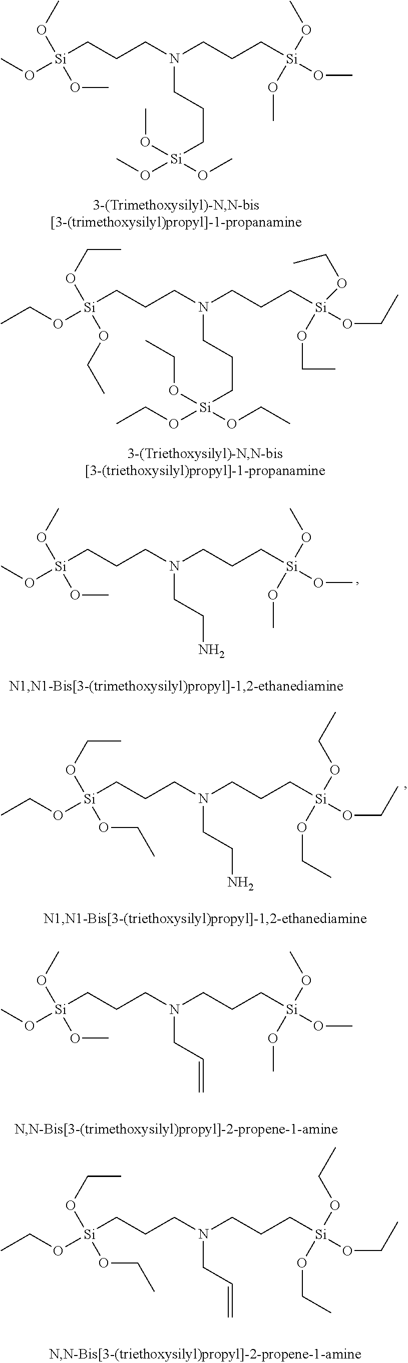

The organic silicon compounds of formula (II) are commercially available.
Bis(trimethoxysilylpropyl)amines with the CAS number 82985-35-1 can be purchased from Sigma-Aldrich®.
Bis[3-(triethoxysilyl)propyl]amines with the CAS number 13497-18-2 can be purchased from Sigma-Aldrich®, for example.
N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich® or Fluorochem®.
3-(triethoxysilyl)-N,N-bis[3-(triethoxysilyl)propyl]-1-propanamine with the CAS number 18784-74-2 can be purchased for example from Fluorochem® or Sigma-Aldrich®.

In a further preferred embodiment, a cosmetic composition according to the present disclosure is exemplified in that the agent (A) is obtained by reacting one or more organic C₁-C₆ alkoxysilanes of formula (II) with water, wherein one or more organic C₁-C₆ alkoxysilanes selected from the group of
3-(Trimethoxysilyl)-N-[3-(Trimethoxysilyl)propyl]-1-propanamine
3-(Triethoxysilyl)-N-[3-(triethoxysilyl)propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl) propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilylpropyl]-1,2-ethanediamine,
N,N-Bis[3-(trimethoxysilyl)propyl]-2-Propen-1-amine and/or
N,N-Bis[3-(triethoxysilyl)propyl]-2-propen-1-amine.

In further dyeing trials, it has also proved to be particularly advantageous, when the agent (A) is obtained by reacting one or more organic C₁-C₆ alkoxysilanes of formula (IV) with water,

(R₉)ₘSi(OR₁₀)ₖ (IV).

The compounds of formula (IV) are organic silicon compounds selected from silanes having one, two or three silicon atoms, wherein the organic silicon compound comprises one or more hydrolysable groups per molecule.

The organic silicon compound(s) of formula (IV) may be designated as silanes of the C₁-C₁₂ alkyl-C₁-C₆ alkoxy silane type (in the case of k=1 to 3) or as silanes of the tetra-C₁-C₆ alkoxy silane type (in the case of k=4),

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the integer 4-k.

In another preferred embodiment, a cosmetic composition according to the present disclosure is exemplified in that the agent (A) is obtained by reacting one or more organic C₁-C₆ alkoxysilanes of formula (IV) with water,

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

In the organic C₁-C₆ alkoxy silanes of formula (IV), the radical R₉ is a C₁-C₁₂ alkyl group or a C₂-C₁₂ alkenyl group. This C₁-C₁₂ alkyl group is saturated and can be linear or branched. The C₂-C₁₂ alkenyl group may comprise one or more double bonds and may be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a C₁-C₆ alkyl group. R₁₀ stands for a methyl group or an ethyl group.

Furthermore, k represents an integer from 1 to 4, and m represents the integer 4-k.

If k stands for the number 4, m is equal to 0. In this case, the silanes of formula (IV) are tetra-C₁-C₆ alkoxy silanes. Suitable silanes of this type are, for example, tetraethoxysilane or tetramethoxysilane.

If k stands for the number 3, m is equal to 1. In this case, the silanes of formula (IV) are C₁-C₁₂ alkyl tri-C₁-C₆ alkoxy silanes.

If k stands for the number 2, m is equal to 2. In this case, the silanes of formula (IV) are di-C₁-C₁₂-alkyl-di-C₁-C₆-alkoxy silanes.

If k stands for the number 1, m is equal to 3. In this case, the silanes of formula (IV) are tri-C₁-C₁₂-alkyl-C₁-C₆-alkoxy silanes.

Dyeing with the best wash fastnesses could be obtained if at least one organic silicon compound of formula (IV) was reacted with water in the preparation of the agent (A), in which the radical k represents the number 3. In this case the radical m stands for the number 1.

Furthermore, particularly satisfactory results were obtained when at least one organic silicon compound of the formula (IV) was used in the preparation of the agent (A), in which the radical R9 is a C1-C8 alkyl group and the radical R10 is a methyl group or an ethyl group.

In another very particularly preferred embodiment, a cosmetic composition according to the present disclosure is exemplified in that the agent (A) is obtained by reacting one or more organic C₁-C₆ alkoxysilanes of the formula (IV) with water,

(R₉)ₘSi(OR₁₀)ₖ (IV),

where
R₉ represents a C₁-C₈ alkyl group,
R₁₀ represents a methyl group or an ethyl group,
k stands for the number 3, and
m stands for the number 1.

Organic silicon compounds of the formula (IV) which are particularly suitable for solving the problem according to the present disclosure are

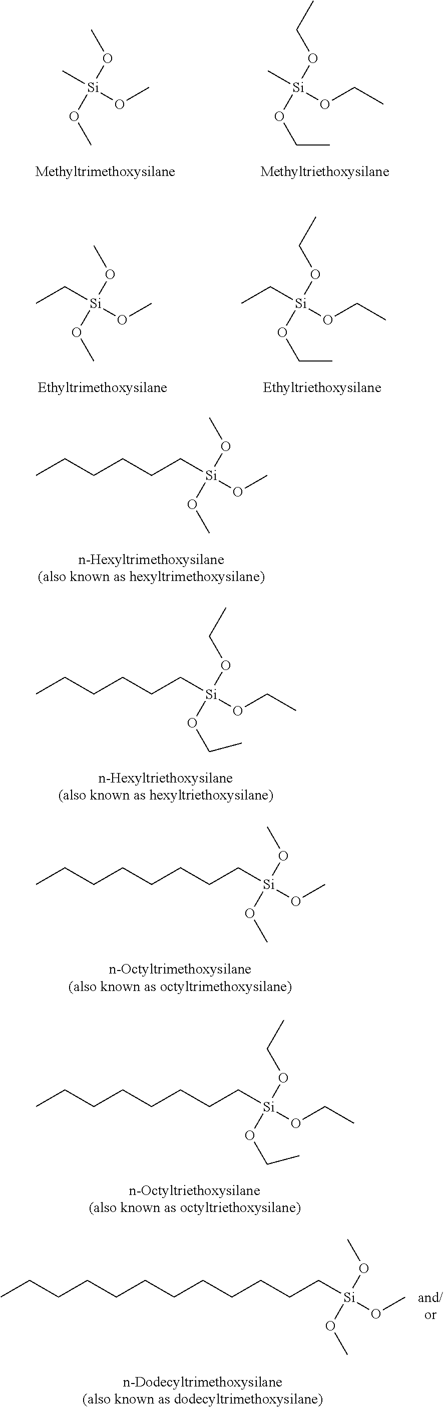

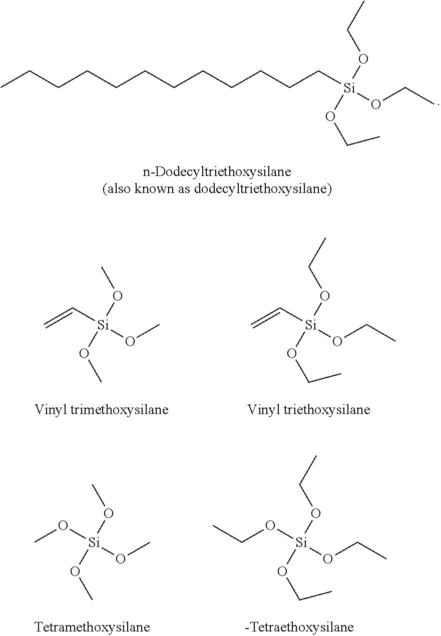

In another preferred embodiment, a cosmetic composition according to the present disclosure is exemplified in that the agent (A) is obtained by reacting one or more organic C₁-C₆ alkoxysilanes of formula (II) with water, wherein one or more organic C₁-C₆ alkoxysilanes selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane and/or
Dodecyltriethoxysilane,
Vinyl trimethoxysilane
Vinyl triethoxysilane
Tetramethoxysilane
Tetraethoxysilane
mixed with a solvent different from water and selectively hydrolyzed and precondensed by adding water and catalyst.

In summary, in a further preferred embodiment, a cosmetic composition according to the present disclosure is exemplified in that the first agent (A) is obtained by reacting one or more organic C₁-C₆ alkoxysilanes selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Vinyl trimethoxysilane
Vinyl triethoxysilane
Tetramethoxysilane
Tetraethoxysilane
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane, and
(2-Dimethylaminoethyl)trimethoxysilane.

In the preparation of the agent (A) from C₁-C₆ alkoxy siloxanes, only one organic C₁-C₆ alkoxysilane from the group of compounds of the formula (I), only one organic C₁-C₆ alkoxysilane from the group of compounds of the formula (II), or also only one organic C₁-C₆ alkoxysilane from the group of compounds of the formula (IV) can be used.

However, agents (A) with particularly advantageous properties were obtained when mixtures of different C₁-C₆ alkoxy-silanes were also used in the preparation of agent (A). Particularly satisfactory results were obtained when both at least one C₁-C₆ organic alkoxysilane of formula (I) and at least one C₁-C₆ organic alkoxysilane of formula (IV) were used. When applied to the keratin material, corresponding agents led to the formation of particularly flexible and resistant coatings or films.

In this context, it was particularly advantageous to use the organic C₁-C₆ alkoxysilanes of the formula (I) and the organic C₁-C₆ alkoxysilanes of the formula (IV) in certain ratios to one another.

In another particularly preferred embodiment, a cosmetic composition according to the present disclosure, exemplified in that the first agent (A) is obtained by mixing one or more organic C₁-C₆ alkoxysilanes of formula (I) and one or more organic C₁-C₆ alkoxysilanes of formula (IV) in a weight ratio of (I)/(IV) of about 1:1 to about 1:10, preferably from about 1:1 to about 1:8, more preferably from about 1:1 to about 1:6, still more preferably from about 1:1 to about 1:4 and most preferably from about 1:2 to about 1:4 to one another and reacted with water.

At a weight ratio of the organic C₁-C₆ alkoxysilanes of the formula (I) and the organic C₁-C₆ alkoxysilanes of the formula (IV), i.e., at a weight ratio (I)/(IV), of about 1:1, for example, 1 part by weight of (3-aminopropyl)triethoxysilane and 1 part by weight of methyltriethoxysilane can be used. Furthermore, 1 part by weight of (3-aminopropyl)triethoxysilane and 1 part by weight of methyltrimethoxysilane can also be used.

At a weight ratio of the organic C₁-C₆ alkoxysilanes of the formula (I) and the organic C₁-C₆ alkoxysilanes of the formula (IV), i.e., at a weight ratio (I)/(IV), of 1:10, for example, 1 part by weight of (3-aminopropyl)triethoxysilane and 10 parts by weight of methyltriethoxysilane can be used. Furthermore, 1 part by weight of (3-aminopropyl)triethoxysilane and 10 parts by weight of methyltrimethoxysilane can also be used.

The stated weight ratios are understood to be the total amount of all organic C₁-C₆ alkoxysilanes of the formula (I) used in the preparation of the agent (A), which is set in relation to the total amount of all organic C₁-C₆ alkoxysilanes of the formula (IV) in the agent (A).

Very preferably, the weight ratio of (I)/(IV) is from about 1:1 to about 1:8, more preferably from about 1:1 to about 1:6, still more preferably from about 1:1 to about 1:4, and most preferably from about 1:2 to about 1:4.

In other words, it has been found to be particularly preferred if the organic C₁-C₆ alkoxysilanes of the formula (IV) are used in a two- to fourfold excess by weight compared with the organic C₁-C₆ alkoxysilanes of the formula (I).

Furthermore, satisfactory results were also obtained when both at least one C₁-C₆ organic alkoxysilane of formula (I) and at least one C₁-C₆ organic alkoxysilane of formula (II) were used. In this context, it was advantageous to use the organic C₁-C₆ alkoxysilanes of the formula (I) and the organic C₁-C₆ alkoxysilane of the formula (II) in certain proportions to one another.

In a further embodiment, a process according to the present disclosure is exemplified in that the cosmetic agent comprises a mixture of organic C₁-C₆ alkoxy-siloxanes obtained by mixing one or more organic C₁-C₆ alkoxysilanes of formula (I) and one or more organic C₁-C₆ alkoxysilanes of formula (II) in a weight ratio of (I)/(II) of about 1:1 to about 1:10, preferably from about 1:1 to about 1:8, more preferably from about 1:1 to about 1:6, still more preferably from about 1:1 to about 1:4 and most preferably from about 1:2 to about 1:4 to each other.

Targeted Hydrolysis of the Organic C₁-C₆ alkoxysilanes in the Agent (A) by Addition of Water.

In the preparation of agent (A), water is added to the mixture of one or more organic C₁-C₆ alkoxysilanes, preferably those of the formula (I), (II) and/or (IV), to initiate selective hydrolysis and, as a result, precondensation.

Water can be added, for example, by dropping or pouring the water into the organic C₁-C₆ alkoxysilane(s). Optionally, a solvent may be present during hydrolysis.

In this case, the dripping or the addition of the water can be done at room temperature. For the application properties of the subsequent cosmetic agent, it can be advantageous if the mixture of organic C₁-C₆ alkoxysilanes and optionally solvent is heated to a temperature of from about 30 to about 80° C., preferably from about 40 to about 75° C., further preferably from about 45 to about 70° C. and very particularly preferably from about 50 to about 65° C., before the water is added.

Adjustment of the preferred and particularly preferred temperature ranges can be accomplished by tempering the reaction vessel or reactor. For example, the reaction vessel or reactor may be surrounded from the outside by a temperature control bath, which may be a water bath or silicone oil bath, for example.

If the reaction is conducted in a double-walled reactor, a temperature-controlled liquid can also be passed through the space formed by the two walls surrounding the reaction chamber.

Since the hydrolysis reaction is exothermic, it has been found to be particularly advantageous to stir or mix the reaction mixture for improved heat dissipation. It is therefore particularly preferred that the water be added while stirring. The reaction, which is now initiated by the addition of water and, if necessary, a catalyst, continues to proceed exothermically, so that the reaction mixture remains at the preferred temperature ranges indicated above or may even heat up further without any further energy being added. It is preferred if the additional heating due to the exothermic nature of the reaction remains within a range of about 5 to about 20° C. If the reaction mixture heats up beyond this range, it is advantageous to cool the mixture.

The water can be added continuously, in partial quantities or directly as a total quantity. To ensure adequate temperature control, the amount and rate of water added is preferentially adjusted. Depending on the amount of silanes used, the addition and reaction can take place over a period of about 2 minutes to about 72 hours.

The addition of the water initiates a selective hydrolysis of the organic C₁-C₆ alkoxysilanes. For the purposes of the present disclosure, targeted hydrolysis means hydrolyzing some, but not all, of the C₁-C₆ alkoxy groups present in the C₁-C₆ organic alkoxysilanes.

The alkoxysilane-water ratio has an influence on the crosslinking within the siloxane network. The degree of crosslinking can be described by so-called T-structures. Here stands T₀ for an unreacted monomer, e.g., methyltriethoxysilane. In the case of a T₁-bond, a siloxane bond exists between two alkoxysilanes, and the two siloxanes are not bonded to any other alkoxysilane. In an T₂-bond, one alkoxysilane is bonded to exactly two others. A T₃-bond describes a siloxane that is bonded to three other siloxanes.

By using different mole ratios of alkoxysilanes to water, the distribution of these different T-structures can be influenced. A high ratio, i.e., less water, leads to less cross-linked structures, whereas a low ratio, i.e., a larger amount of water, leads to more cross-linking.

The quantitative determination of the T0 structures, T1 structures, T2 structures and T3 structures contained in the agent in each case can be conducted, for example, by employing quantitative 29Si NMR spectroscopy.
Use of NMR sample tubes
Device: Agilent®, 600 MHz

29Si-NMR spectra were recorded in chloroform from each of the compositions. Measurements were taken on the day of production and after a defined period.
Standard: TMS (tetramethylsilane)
Relaxation accelerator: Chromium(III) acetylacetonate.

By using the relaxation accelerator, the integrals of the individual signals became comparable with each other. The sum over all integrals was set equal to 100 mol %. For the quantitative determination, the area of each individual signal was related to the total sum over all integrals.

For example, the spectra can be measured using the procedure described in Journal of Organometallic Chemistry 625 (2001), 208-216.

The amount of water necessary to prepare the agent (A) is equal to the molar amount of water, which is determined by equation (G-1)

X=[(n_{I}(Alkoxysilane)×n_{II}(Alkoxy groups)]/n(H₂O) (G-1)

where
n(H₂O) is the molar amount of water used in the agent (A),
n_{I} is the molar amount of the organic C₁-C₆ alkoxysilanes used in the agent (A),
n_{II} is the number of C₁-C₆ alkoxy groups per C₁-C₆ organic alkoxysilane used in the agent (A), and
X is a number from about 3.0 to about 100, and

In other words, the index number X indicates the molar ratio of the total number of moles of hydrolysable C₁-C₆ alkoxy groups to the molar amount of water used.

nI indicates the total molar amount of C₁-C₆ organic alkoxysilanes used in the agent (A). If only one C₁-C₆ alkoxysilane of a certain structure is used, the total molar amount n_{I} corresponds to the molar amount of the C₁-C₆ alkoxysilane used.

However, if a mixture of C₁-C₆ alkoxysilanes is used to prepare the agent (A), the total molar amount is the sum of the individual molar amounts of each C₁-C₆ alkoxysilane used.

Furthermore, nII indicates the number of C₁-C₆ alkoxy groups per C₁-C₆ organic alkoxysilane used in the agent (A). If only one C₁-C₆ alkoxysilane of a given structure is used, n_{II} corresponds to the number of C₁-C₆ alkoxy groups present in that molecule.

However, if a mixture of C₁-C₆ alkoxysilanes is used to prepare the agent (A), the number of C₁-C₆ alkoxy groups of each C₁-C₆ alkoxysilane enters the equation.

If several C₁-C₆ alkoxysilanes are used to prepare the agent (A), the above equation expands to equation (G-1′) by forming the respective summands:

$$X = \frac{\sum [ nI ( Alkoxysilan ) \times nII ( Alkoxygruppen ) ]}{n ( H 2 O )} \qquad ( G - {1}^{′} )$$

### Calculation Example 1

In the preparation of agent (A), 23.52 g of 3-aminopropyltriethoxsilane (C₉H₂₃NO₃Si=221.37 g/mol) and 47.07 g of methyltriethoxysilane (C₇H₁₈O₃Si=178.34 g/mol) were mixed. To this mixture, 25 g of ethanol (abs.) was added with stirring. Then, 6.06 g of water (18.015 g/mol) was added with stirring.
23.52 g 3-aminopropyltriethoxsilane (AMEO)=0.106 mol
3-Aminopropyltriethoxsilane has 3 hydrolysable alkoxy groups per molecule.
47.07 g methyltriethoxysilane (MTES)=0.264 mol
Methyltrimethoxysilane has 3 hydrolysable alkoxy groups per molecule).
6.06 g water=0.336 mol

When two different C₁-C₆ alkoxysilanes are used, the value X is calculated by applying the formula (G-1′) under formation of appropriate sums, i.e.

$$X = \frac{[ n ( AMEO ) \times 3 ] + ( n ( MTES ) \times 3 ]}{n ( H 2 O )}$$

$$X = \frac{[ ( 0 , 106 ) \times 3 ] + [ ( 0 , 264 ) \times 3 ]}{0 , 336} = 3.3$$

### Calculation Example 2

In the preparation of agent (A), 23.95 g of 3-aminopropyltriethoxsilane (C₉H₂₃NO₃Si=221.37 g/mol) and 47.90 g of methyltriethoxysilane (C₇H₁₈O₃Si=178.34 g/mol) were mixed. To this mixture, 30 g of ethanol (abs.) was added with stirring. Then, 4.13 g of water (18.015 g/mol) was added with stirring.
23.95 g 3-aminopropyltriethoxsilane (AMEO)=0.1082 mol
3-Aminopropyltriethoxsilane has 3 hydrolysable alkoxy groups per molecule.
47.90 g methyltriethoxysilane (MTMS)=0.269 mol
Methyltrimethoxysilane has 3 hydrolysable alkoxy groups per molecule).
4.13 g water=0.229 mol

When two different C₁-C₆ alkoxysilanes are used, the value X is calculated by applying the formula (G-1′) under formation of appropriate sums, i.e.

$$X = \frac{[ n ( AMEO ) \times 3 ] + ( n ( MTES ) \times 3 ]}{n ( H 2 O )}$$

$$X = \frac{[ ( 0 , 1082 ) \times 3 ] + [ ( 0 , 269 ) \times 3 ]}{0 , 229} = 4.9$$

If further or other mixtures of C₁-C₆ alkoxy silanes are used, the formula (G-1) or (G-1′) is adapted accordingly or extended by the corresponding summands.

The degree of crosslinking set during the preparation of the agent (A) by adding the appropriate amount of water influences the application properties of the coating produced during subsequent application to the keratin material. A particularly stable, and resistant film could be produced when first agent (A) is obtained by reacting with an amount of water equal to the molar amount of water determined by equation (G-1), where X is equal to a number from about 3.5 to about 85.0, preferably from about 3.7 to about 65.0, more preferably from about 3.9 to about 45.0, still more preferably from about 4.1 to about 25.9, and most preferably from about 4.3 to about 5.0.

In the context of a further particularly preferred embodiment, a cosmetic composition according to the present disclosure is therefore exemplified in that the first agent (A) is obtained by reaction with an amount of water corresponding to the molar amount of water determined according to equation (G-1), where X is equal to a number from about 3.5 to about 85.0, preferably from about 3.7 to about 65.0, more preferably from about 3.9 to about 45.0, still more preferably from about 4.1 to about 25.9, and most preferably from about 4.3 to about 5.0.

### Agent (b)

Analogous to agent (A), agent (B) is also obtained by reacting one or more organic C₁-C₆ alkoxysilanes with a certain amount of water. When preparing the agent (B), the amount of water used (calculated in moles of water) is given by formula (G-2). However, compared to the agent (A), a higher amount of water is used in the production of the agent (B). Agent (B) may also be referred to as a silane blend and comprises a mixture of C₁-C₆ alkoxysilanes reacted to form siloxanes. However, due to the different amount of water used, the molecular weight distribution and oligomer structure in the agent (B) is different from that in the agent (A).

In summary, therefore, in principle, the method of preparation of the agents (A) and (B) can be chosen to be the same. The difference lies in the different amounts of water required to produce agents (A) and (B). Less water is used in the production of agent (A), whereas the amount of water used in the production of agent (B) is higher.

Organic C₁-C₆ Alkoxy Silanes in the Agent (B)

In particular, the organic C₁-C₆ alkoxy silanes already described in detail in connection with the preparation of the agent (A) can be used in the preparation of the agent (B). Therefore, explicit reference is made at this point to the section “Organic C₁-C₆ alkoxy silanes in the agent (A)”. The organic C₁-C₆ alkoxy silanes described as preferred or particularly preferred in the preparation of agent (A) are also preferred or particularly preferred in the preparation of agent (B).

In the preparation of agent (B), the same organic C₁-C₆ alkoxy silanes can be used that are used in the preparation of agent (A). However, it is also possible and according to the present disclosure if the agent (B) is obtained by reacting organic C₁-C₆ alkoxy silanes different from those used in the agent (A).

Preferably, the same organic C₁-C₆ alkoxy silanes are used in agents (A) and (B).

In summary, therefore, in the context of a further particularly preferred embodiment, a cosmetic composition according to the present disclosure is exemplified in that the second agent (B) is obtained by reacting one or more organic C₁-C₆ alkoxysilanes of formula (I) and/or (II) and/or (IV) with water,

R₁R₂N-L-Si(OR₃)ₐ(R₄)_{b} (I)

where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a stands for an integer from 1 to 3, and
b stands for the integer 3-a, and

(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),

(A″″)-Si(R₆″)_{d}″(OR₅″)_{c}″ (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h radicals are different from 0,

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

Further summarized, a cosmetic composition according to the present disclosure is exemplified in that the second agent (B) is obtained by reacting one or more organic C₁-C₆ alkoxysilanes selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Vinyl trimethoxysilane
Vinyl triethoxysilane
Tetramethoxysilane
Tetraethoxysilane
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane, and
(2-Dimethylaminoethyl)trimethoxysilane.

Also, in the preparation of the agent (B), either only a C₁-C₆ organic alkoxysilane from the group of compounds of the formula (I), only a C₁-C₆ organic alkoxysilane from the group of compounds of the formula (II), or also only a C₁-C₆ organic alkoxysilane from the group of compounds of the formula (IV) can be used.

In the preparation of the agent (B), the mixtures of organic C₁-C₆ alkoxysilanes which have also been mentioned as preferred in connection with the preparation of the agent (A) can preferably also be used.

Targeted Hydrolysis of the Organic C₁-C₆ alkoxysilanes in the Agent (B) by Addition of Water.

In the preparation of agent (B), water is added to the mixture of one or more organic C₁-C₆ alkoxysilanes, preferably those of the formula (I) and/or (II) and/or (IV), to initiate selective hydrolysis and, as a result, precondensation. Compared to the agent (A), the amount of water used in the production of the agent (B) is higher.

Water can be added, for example, by dropping or pouring the water into the mixture of the C₁-C₆ organic alkoxysilanes and the solvent.

The addition of water in the preparation of the agent (B) can be conducted in the same way as in the preparation of the agent (A).

As with agent (A), the degree of crosslinking in agent (B) is influenced using different molar ratios between alkoxysilanes and water. A high ratio, i.e., less water, leads to less cross-linked structures, whereas a low ratio, i.e., a larger amount of water, leads to stronger cross-linking.

As with agent (A), the alkoxysilane-water ratio also influences crosslinking within the siloxane network in agent (B). The degree of crosslinking can be described by so-called T-structures. Here T₀ stands for an unreacted monomer, e.g., methyltriethoxysilane. In the case of a T₁-bond, a siloxane bond exists between two alkoxysilanes, and the two siloxanes are not bonded to any other alkoxysilane. In an T₂-bond, one alkoxysilane is bonded to exactly two others. A T₃-bond describes a siloxane that is bonded to three other siloxanes.

By using different mole ratios between alkoxysilanes and water, the distribution of these different T-structures can be influenced. A high ratio, i.e., less water, leads to less cross-linked structures, whereas a low ratio, i.e., a larger amount of water, leads to stronger cross-linking.

The quantitative determination of the T0 structures, T1 structures, T2 structures and T3 structures contained in the agent in each case can be conducted, for example, by employing quantitative 29Si NMR spectroscopy.
Use of NMR sample tubes
Device: Agilent®, 600 MHz
29Si-NMR spectra were recorded in chloroform from each of the compositions. Measurements were taken on the day of production and after a defined period.
Standard: TMS (tetramethylsilane)
Relaxation accelerator: Chromium(III) acetylacetonate
By using the relaxation accelerator, the integrals of the individual signals became comparable with each other. The sum over all integrals was set equal to 100 mol %.
For the quantitative determination, the area of each individual signal was related to the total sum over all integrals.

The amount of water necessary to prepare the agent (B) is equal to the molar amount of water, which is determined according to equation (G-2)

Y=[(nₐ(Alkoxysilane)×n_{b}(Alkoxy groups)]/m(H₂O) (G-2)
where
m(H₂O) is the molar amount of water used in the agent (B),
nₐ is the molar amount of the organic C₁-C₆ alkoxysilanes used in the agent (B),
n_{b} is the number of C₁-C₆ alkoxy groups per C₁-C₆ organic alkoxysilane used in the agent (B), and
Y is a number from about 0.1 to about 2.9.

In other words, the index number Y indicates the molar ratio from the total number of moles of hydrolysable C₁-C₆ alkoxy groups, which is related to the molar amount of water used.

nₐ indicates the total molar amount of C₁-C₆ organic alkoxysilanes used in the agent (B). If only a C₁-C₆ alkoxysilane of a certain structure is used, the total molar amount nₐ corresponds to the molar amount of the C₁-C₆ alkoxysilane used.

However, if a mixture of C₁-C₆ alkoxysilanes is used to prepare the agent (B), the total molar amount is the sum of the individual molar amounts of each C₁-C₆ alkoxysilane used.

Furthermore, n_{b} indicates the number of C₁-C₆ alkoxy groups per C₁-C₆ organic alkoxysilane used in the agent (B). If only one C₁-C₆ alkoxysilane of a given structure is used, nb corresponds to the number of C₁-C₆ alkoxy groups present in that molecule.

However, if a mixture of C₁-C₆ alkoxysilane is used to prepare the agent (B), the number of C₁-C₆ alkoxy groups of each C₁-C₆ alkoxysilane enters the equation.

If several C₁-C₆ alkoxysilanes are used to prepare the agent (B), the above equation expands to equation (G-2′) by forming the respective summands:

$$Y = \frac{\sum [ na ( Alkoxysilan ) \times nb ( Alkoxygruppen ) ]}{m ( H 2 O )} \qquad ( G - {2}^{′} )$$

### Calculation Example 3

In the preparation of agent (B), 23.05 g of 3-aminopropyltriethoxsilane (C₉H₂₃NO₃Si=221.37 g/mol) and 46.11 g of methyltriethoxysilane (C₇H₁₈O₃Si=178.34 g/mol) were mixed. To this mixture, 25 g of ethanol (abs.) was added with stirring. Then, 8.34 g of water (18.015 g/mol) was added with stirring.
23.05 g 3-aminopropyltriethoxsilane (AMEO)=0.1041 mol
3-Aminopropyltriethoxsilane has 3 hydrolysable alkoxy groups per molecule.
46.11 g methyltriethoxysilane (MTES)=0.259 mol
Methyltrimethoxysilane has 3 hydrolysable alkoxy groups per molecule).
8.34 g water=0.463 mol

When two different C₁-C₆ alkoxysilanes are used, the value Y is calculated by applying formula (G-2′) under formation of corresponding sums, i.e.

$$Y = \frac{[ n ( AMEO ) \times 3 ] + ( n ( MTES ) \times 3 ]}{m ( H 2 O )}$$

$$y = \frac{[ ( 0 , 1041 ) \times 3 ] + [ ( 0 , 259 ) \times 3 ]}{0 , 463} = 2.61$$

If further or other mixtures of C₁-C₆ alkoxy silanes are used, the formula (G-2) or (G-2′) is adapted accordingly or extended by the corresponding summands.

The degree of crosslinking set during the preparation of the agent (B) by adding the appropriate amount of water influences the application properties of the coating produced during subsequent application to the keratin material. A particularly stable, and resistant film could be produced when the second agent (B) is obtained by reacting with an amount of water equal to the molar amount of water determined by equation (G-2), where Y is equal to a number from about 0.4 to about 2.8, preferably from about 0.7 to about 2.7, more preferably from about 1.0 to about 2.6, still more preferably from about 1.3 to about 2.5, and most preferably from about 1.7 to about 2.4.

In another particularly preferred embodiment, a cosmetic composition according to the present disclosure is therefore exemplified in that the second agent (B) is obtained by reaction with an amount of water corresponding to the molar amount of water determined according to equation (G-2), wherein Y is equal to a number from about 0.4 to about 2.8, preferably from about 0.7 to about 2.7, more preferably from about 1.0 to about 2.6, still more preferably from about 1.3 to about 2.5, and most preferably from about 1.7 to about 2.4.

Reaction of the Organic C₁-C₆ alkoxy silanes with Water in Agents (A) and (B)

In agents (A) and (B), the reaction of the organic C₁-C₆ alkoxy silanes with water can take place in diverse ways. The reaction starts as soon as the C₁-C₆ alkoxy silanes meet water by mixing. As soon as C₁-C₆ alkoxy silanes and water come into contact, an exothermic hydrolysis reaction takes place according to the following scheme (reaction scheme using the example of 3-aminopropyltriethoxysilane):

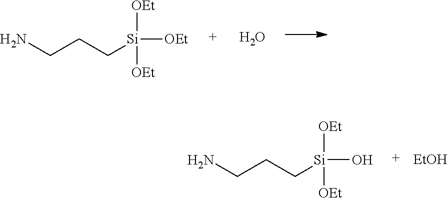 [SMILES: "EtOH" ; CCO[Si](CCCN)(OCC)OCC ; O ; CCO[Si](O)(CCCN)OCC]

Depending on the number of hydrolysable C₁-C₆ alkoxy groups per silane molecule, the hydrolysis reaction can also occur several times per C₁-C₆ alkoxy silane used:

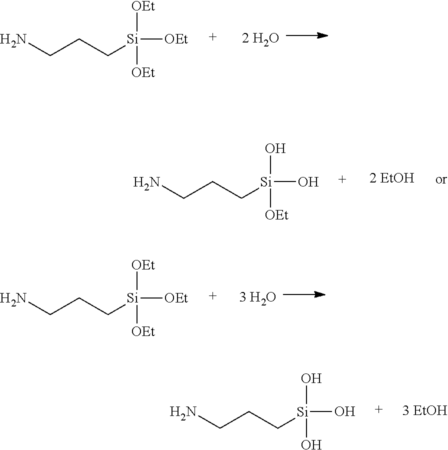

Hydrolysis using the example of methyltrimethoxysilane:

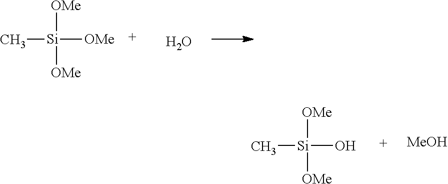

Depending on the amount of water used, the hydrolysis reaction can also take place several times per C₁-C₆ alkoxy silane used:

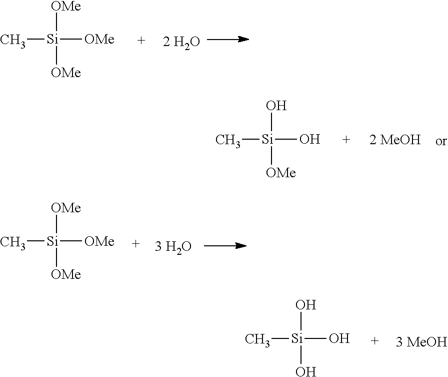

Following the hydrolysis or quasi simultaneously with the hydrolysis, condensation of the partially (or in parts completely) hydrolyzed C₁-C₆ alkoxy silanes takes place. The precondensation can proceed, for example, according to the following scheme:

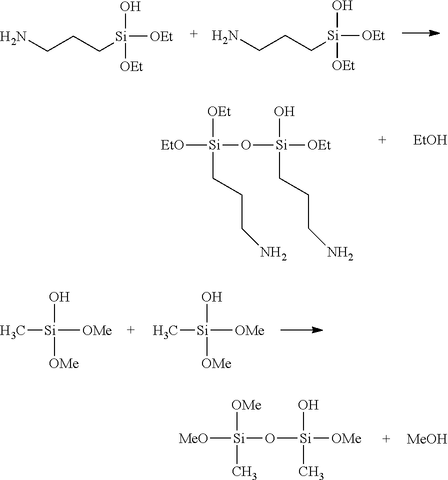

Both partially hydrolyzed and fully hydrolyzed C₁-C₆ alkoxysilanes can participate in the condensation reaction, undergoing condensation with not yet reacted, partially or also fully hydrolyzed C₁-C₆ alkoxysilanes.

Condensation reactions include (shown using the mixture (3-aminopropyl)triethoxysilane and methyltrimethoxysilane):

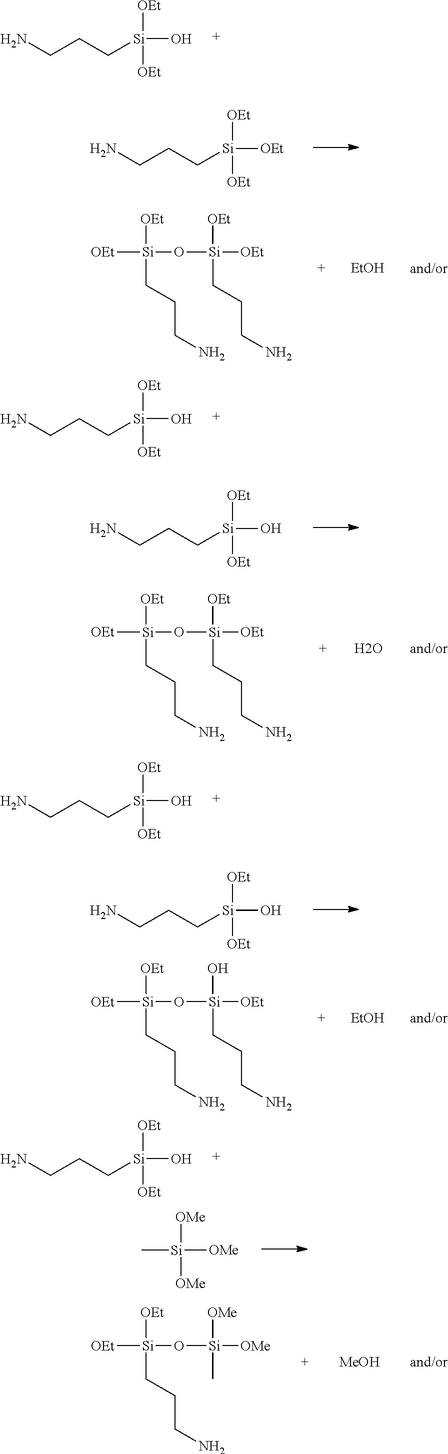

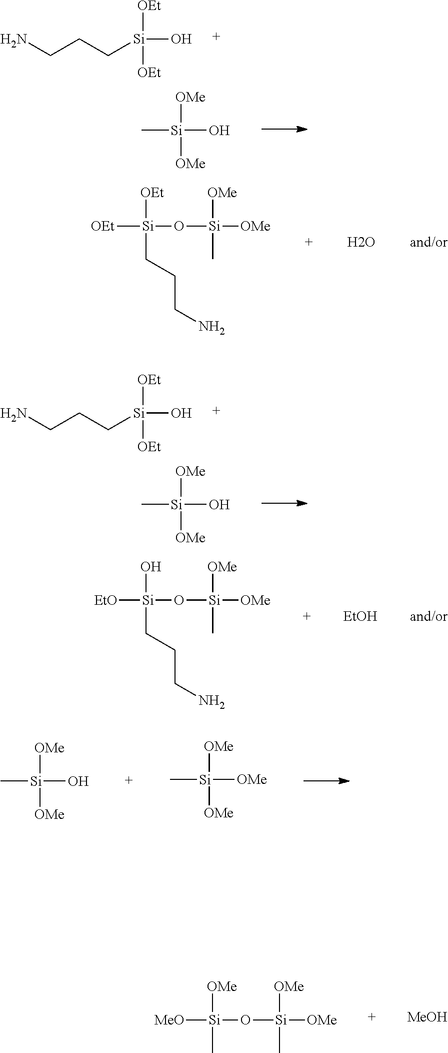

In the above exemplary reaction schemes, the condensation to a dimer is shown in each case, but further condensations to oligomers with several silane atoms are also possible and preferred, since they lead to the crosslinking of the siloxane mixture described above.

### Reaction Vessels

Agents (A) and (B) are preferably prepared from organic C₁-C₆ alkoxysiloxanes in a reactor or reaction vessel suitable for this purpose. A reaction vessel that is very suitable for smaller preparations is, for example, a glass flask commonly used for chemical reactions with a capacity of 1 liter, 3 liters or 5 liters, such as a 3-liter single-neck or multi-neck flask with ground joints.

A reactor is a confined space (container, vessel) that has been specially designed and manufactured to allow certain reactions to take place and be controlled under defined conditions.

For larger approaches, it has proven advantageous to conduct the reaction in reactors made of metal. Typical reactors may include, for example, a 10-liter, 20-liter, or 50-liter capacity. Larger reactors for the production area can also include fill volumes of 100-liters, 500-liters, or 1000-liters.

Double-wall reactors have two reactor shells or reactor walls, with a tempering fluid circulating in the area between the two walls. This enables particularly good adjustment of the temperature to the required values.

The use of reactors, in particular double-walled reactors with an enlarged heat exchange surface, has also proven to be particularly suitable, whereby the heat exchange can take place either through internal installations or using an external heat exchanger.

Corresponding reactors are, for example, laboratory reactors from the company IKA®. In this context, the models “LR-2.ST” or the model “magic plant” can be mentioned.

Other reactors that can be used are reactors with thin-film evaporators, since this allows particularly good heat dissipation and thus particularly precise temperature control. Thin film evaporators are alternatively referred to as thin film evaporators. Thin film evaporators can be purchased commercially from Asahi Glassplant® Inc. for example.

### Mixing the Agents (A) and (B)

The cosmetic composition according to the present disclosure is now obtained by mixing a first agent (A) with a second agent (B).

According to the present disclosure, this mixing takes place before the cosmetic composition is applied to the keratin material. Here, the mixing of agents (A) and (B) can either take place shortly before use, or the two agents (A) and (B) can, for example, first be produced separately, then stored for a certain period, and then mixed. After this mixing process, the cosmetic preparation can be stored again for a certain period before it is then applied to the keratin material.

Agents (A) and (B) may first be stored for a few days, weeks, or months, for example, before being mixed. Also, the cosmetic preparation prepared by mixing agents (A) and (B) can be stored again for some time, for example, for days, weeks, or months. It has been found particularly preferable to fill the cosmetic composition, after mixing agents (A) and (B), into a small container, such as a bottle, tube, sachet, or jar, which is suitable for end-user application and can be applied to the hair, for example, by the user as part of a hair treatment.

The degree of crosslinking in the cosmetic composition according to the present disclosure also depends on the mixing ratio in which the two agents (A) and (B) are mixed. To produce particularly stable, resistant, and reproducible films or coatings on the keratin material, it has proved particularly preferable to mix the agents (A) and (B) in a specific ratio.

Particularly good results could be obtained when the agent (A) and the agent (B) were mixed together in a weight ratio (A)/(B) of from about 1:5 to about 5:1, preferably from about 1:4 to about 4:1, more preferably from about 1:3 to about 3:1, still more preferably from about 1:1 to about 3:1, and most preferably from about 2:1 to about 3:1.

In the context of a particularly preferred embodiment, a cosmetic composition according to the present disclosure is exemplified in that it is obtained by mixing the first agent (A) with the second agent (B) in a weight ratio (A)/(B) of from about 1:5 to about 5:1, preferably from about 1:4 to about 4:1, more preferably from about 1:3 to about 3:1, still more preferably from about 1:1 to about 3:1, and most preferably from about 2:1 to about 3:1.

### Sample Calculation

First, both agents (A) and (B) were prepared as described previously, filled into separate containers, and then stored for 3 months. Then 23 g of agent (A) was mixed with 10 g of agent (B).
The weight ratio (A)/B) is 23/10 and thus 2.3:1.
This mixture of (A) and (B) was applied to hair 10 min after completion of shaking.

### Sample Calculation

First, the two agents (A) and (B) were prepared, filled into separate containers, and then stored for 14 days. Then 50 g of agent (A) was mixed with 20 g of agent (B).
The weight ratio (A)/B) is 50/20 and thus 2.5:1.
This mixture of (A) and (B) was then filled back into a container and stored until use.

Mixing of agents (A) and (B) can be done, for example, by stirring or by shaking. Depending on the batch size of the agents (A) and (B) produced, mixing can be conducted in a smaller vessel, such as a flask or beaker, or—for larger batches—in a reactor.

Also, in the cosmetic composition according to the present disclosure, the quantitative determination of the T0 structures, T1 structures, T2 structures and T3 structures contained in each case can be conducted by quantitative 29Si NMR spectroscopy.

### Solvent

In the preparation of the agents (A) and (B) by reaction of the organic C₁-C₆ alkoxy-siloxanes with water, it is preferred if the organic C₁-C₆ alkoxysilane or organic C₁-C₆ alkoxysilanes, the above-mentioned preferred and especially preferred representatives, are mixed in the first step with a solvent other than water.

Mixing can be accomplished, for example, by first placing the solvent other than water in a suitable reactor or reaction vessel and then adding the organic C₁-C₆ alkoxysilane(s). The addition can be done by dripping or pouring. Furthermore, it is also possible and in accordance with the present disclosure if at least one organic C₁-C₆ alkoxysilane is first introduced into the reaction vessel and then the solvent is added or added dropwise.

A sequential approach is also possible, i.e., first the addition of solvent and a first organic C₁-C₆ alkoxysilane, then again the addition of a solvent and then again the addition of another organic C₁-C₆ alkoxysilane.

The solvent is preferably added with stirring.

It may be preferred to select a solvent that has a boiling point at normal pressure (1013 hPa) of from about 20 to about 90° C., preferably from about 30 to about 85° C., and most preferably from about 40 to about 80° C.

Suitable Solvents Include:
Dichloromethane with a boiling point of 40° C. (1013 mbar)
Methanol with a boiling point of 65° C. (1013 mbar)
Tetrahydrofuran with a boiling point of 65.8° C. (1013 mbar)
Ethanol with a boiling point of 78° C. (1013 mbar)
Isopropanol with a boiling point of 82° C. (1013 mbar)
Acetonitrile with a boiling point of 82° C. (1013 mbar)

Furthermore, very particularly preferred solvents can be selected from the group of monohydric or polyhydric C₁-C₁₂ alcohols. Monohydric or polyhydric C₁-C₁₂ alcohols are compounds containing one to twelve carbon atoms and bearing one or more hydroxyl groups. Other functional groups different from the hydroxy groups are not present in the C₁-C₁₂ alcohols according to the present disclosure. The C₁-C₁₂ alcohols can be aliphatic or aromatic.

Suitable C₁-C₁₂ alcohols may include methanol, ethanol, n-propanol, isopropanol, n-pentanol, n-hexanol, benzyl alcohol, 2-phenylethanol, 1,2-propanediol, 1,3-propanediol and glycerol. Particularly suitable C₁-C₁₂ alcohols are methanol, ethanol, and isopropanol.

In another particularly preferred embodiment, a cosmetic composition according to the present disclosure is exemplified in that
the first agent (A) is obtained by reacting one or more organic C₁-C₆ alkoxysilanes with water in the presence of a solvent selected from the group of methanol, ethanol, and isopropanol, and/or
the second agent (B) is obtained by reacting one or more organic C₁-C₆ alkoxysilanes with water in the presence of a solvent selected from the group of methanol, ethanol, and isopropanol.

After the mixture of organic C₁-C₆ alkoxy siloxanes has been prepared, the solvent can be removed again. Removal can be accomplished by distillation off under reduced pressure, for example using a rotary evaporator. However, further work has shown that it may also be advantageous to leave the solvent(s) in the mixture of C₁-C₆ organic alkoxy siloxanes.

### Catalyst

In the preparation of the agents (A) and (B), one or more organic C₁-C₆ alkoxy silanes are reacted with water, hydrolysis and precondensation of the C₁-C₆ alkoxy silanes taking place. This reaction particularly preferably takes place in the presence of a catalyst, with the addition of the catalyst having the effect of initiating or accelerating the hydrolysis reaction.

By a catalyst, the skilled person understands a substance that increases the reaction rate by lowering the activation energy of a chemical reaction without itself being consumed.

The catalyst can be added before or after the water is added.

For the preparation of mixtures of organic C₁-C₆ alkoxy siloxanes, it has proved particularly advantageous to use a catalyst that can be dissolved or dispersed in water and is then added to the mixture of organic C₁-C₆ alkoxy silanes and solvent together with the water as a solution or dispersion.

Very preferably, the catalyst is selected from the group of inorganic or organic acids and inorganic or organic bases.

Particularly well-suited catalyst types are inorganic and organic acids, which can preferably be selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, and 1-hydroxyethane-1,1-diphosphonic acid. Explicitly, sulfuric acid, hydrochloric acid and maleic acid are particularly preferred.

Other particularly suitable catalysts are inorganic and organic bases, which can preferably be selected from the group of sodium hydroxide, potassium hydroxide, magnesium hydroxide and calcium hydroxide. Sodium hydroxide and potassium hydroxide are particularly preferred.

Other bases that can be used include ammonia, alkanolamines and/or basic amino acids.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, and 2-amino-2-methylpropan-1,3-diol.

For the purposes of the present disclosure, an amino acid is an organic compound containing at least one protonatable amino group and at least one —COOH or —SO₃H group in its structure. Preferred amino acids are aminocarboxylic acids, especially α-(alpha)-aminocarboxylic acids and ω-aminocarboxylic acids, whereby α-aminocarboxylic acids are particularly preferred.

According to the present disclosure, basic amino acids are those amino acids which have an isoelectric point pI of greater than about 7.0.

Basic α-aminocarboxylic acids contain at least one asymmetric carbon atom. In the context of the present disclosure, both enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine, and histidine, especially preferably arginine and lysine. In another particularly preferred embodiment, an agent according to the present disclosure is therefore exemplified in that the alkalizing agent is a basic amino acid from the group arginine, lysine, ornithine and/or histidine.

In addition, other inorganic alkalizing agents or bases can also be used. Inorganic alkalizing agents that can be used according to the present disclosure can be selected, for example, from the group formed by sodium phosphate, potassium phosphate, sodium silicate, sodium metasilicate, potassium silicate, sodium carbonate and potassium carbonate.

In another very particularly preferred embodiment, a process according to the present disclosure is exemplified in that the catalyst is selected from the group of inorganic and organic bases, preferably from the group of sodium hydroxide, potassium hydroxide, magnesium hydroxide and calcium hydroxide.

In another very particularly preferred embodiment, a cosmetic composition according to the present disclosure is exemplified in that
the first agent (A) is obtained by reacting one or more organic C₁-C₆ alkoxysilanes with water in the presence of a catalyst selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid and 1-hydroxyethane-1,1-diphosphonic acid, sodium hydroxide, potassium hydroxide, magnesium hydroxide and calcium hydroxide, and/or
the second agent (B) is obtained by reacting one or more organic C₁-C₆ alkoxysilanes with water in the presence of a catalyst selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid and 1-hydroxyethane-1,1-diphosphonic acid, sodium hydroxide, potassium hydroxide, magnesium hydroxide and calcium hydroxide.

According to the present disclosure, the catalysts are preferably used in the usual quantity ranges for catalysts. Since the catalysts accelerate the hydrolysis or condensation without being consumed themselves, the quantities used can be chosen to be correspondingly low.

Thus, the catalyst or catalysts can be used in an amount range from about 0.0000001 to about 2.0 wt. %, preferably from about 0.0001 to about 1.5 wt. % and most preferably from about 0.01 to about 1.0 wt. % in the agent (A) and/or (B). Here, the figure in wt. % refers to the total amount of catalysts used, which is set in relation to the total amount of organic C₁-C₆ alkoxy siloxanes plus solvent plus water used in the respective agent.

### Process for the Preparation of Agents (A) and (B)

In principle, various methods are conceivable for producing agents (A) and (B).

For example, one manufacturing process is as follows:
i) A quantity of solvent, for example ethanol or methanol, and a quantity of organic C₁-C₆ alkoxysilane, for example methyltrimethoxysilane or methyltriethoxysilane, are placed in a round bottom flask.
ii) The filled round bottom flask is equipped with a stirrer and a thermometer.
iii) Then the round bottom flask is clamped into a stirring apparatus and connected to the cooling system.
iv) The flask contents are brought to the desired temperature by employing an oil bath while stirring at about 500 rpm.
v) When the desired temperature is reached, the amount of water with catalyst is dosed into the round bottom flask using a 100 ml dropping funnel over 3 minutes. The dropping funnel is removed from the apparatus and replaced by a new dropping funnel containing the previously calculated amount of another organic C₁-C₆ alkoxysilane, for example (3-aminopropyl)-triethoxysilane.
vi.) About 10 to about 60 minutes after completion of the addition of water plus catalyst, the second organic C₁-C₆ alkoxysilane is added.
vii.) It is stirred for about another 30 to about 240 minutes.
viii) The mixture of organic C₁-C₆-alkoxy-siloxanes prepared in this way is filled into a tight container while still hot.

This production process is particularly well suited when at least one acid, for example an acid selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, and 1-hydroxyethane-1,1-diphosphonic acid, is used as the catalyst.

Furthermore, this production process is also particularly suitable if at least one base, preferably from the group of sodium hydroxide, potassium hydroxide, magnesium hydroxide and calcium hydroxide, is used as catalyst.

As this production process shows, it is in accordance with the present disclosure and, in the case of the use of acids or bases as catalysts, even particularly preferred if the mixing of a first organic C₁-C₆-alkoxysilane with the solvent and the addition of water and catalyst, which initiate the hydrolysis and precondensation of the first C₁-C₆-alkoxysilane, are followed additionally by the addition of a second organic C₁-C₆-alkoxysilane.

Another manufacturing process is the following:
i.) A quantity of solvent, for example ethanol or methanol, and a quantity of organic C₁-C₆ alkoxysilanes, for example methyltrimethoxysilane and/or methyltriethoxysilane and/or (3-aminopropyl)-triethoxysilane, are placed in a round bottom flask. Particularly preferred in this step are a mixture of methyltrimethoxysilane and (3-aminopropyl)-triethoxysilane, a mixture of methyltriethoxysilane and (3-aminopropyl)-triethoxysilane, or a mixture of ethyltriethoxysilane and (3-aminopropyl)-triethoxysilane.
ii.) The filled round bottom flask is equipped with a stirrer and a thermometer.
iii.) Then the round-bottom flask is clamped into a stirring apparatus and connected to the cooling system.
iv.) The contents of the flask are brought to the desired temperature by employing an oil bath while stirring at about 500 rpm.
v.) When the desired temperature is reached, the amount of water with catalyst is dosed into the round bottom flask using a 100 ml dropping funnel over about 3 minutes.
vi.) It is stirred for about another 30 to about 240 minutes.
vii.) The mixture of organic C₁-C₆ alkoxy siloxanes produced in this way is filled into a tight container while still hot.
This production process is particularly well suited when at least one base, for example a base selected from the group of sodium hydroxide, potassium hydroxide, magnesium hydroxide and calcium hydroxide, is used as the catalyst.

### Multi-Component Packaging Unit (Kit-of-Parts)

The cosmetic composition of the first subject matter of the present disclosure shows particularly good suitability for the treatment of keratinous materials, whereby a treatment can be understood to mean, for example, care, conditioning, shaping or even hairstyling. The cosmetic composition is particularly suitable for coloring the cosmetic material.

To coloring, the formation of the film or coating is preferably conducted in the presence of a coloring compound, in the presence of a pigment and/or a direct dye. In this case, it is particularly advantageous if the colorant compounds are provided to the user in a separate, separately assembled packaging unit.

A second object of the present disclosure is a multi-component packaging unit (kit-of-parts) for dyeing keratinous material, in particular human hair, which comprises separately assembled
a first packaging unit containing a cosmetic composition as already disclosed in detail in the description of the first subject matter of the present disclosure, and
a second packaging unit containing an agent (C), the agent (C) comprising at least one pigment and/or a direct dye, and
optionally a third packaging unit containing an agent (D), the agent (D) comprising at least one film-forming polymer.

For reasons of storage stability, the cosmetic composition prepared by mixing agents (A) and (B) preferably contains no other cosmetic ingredients. However, the separately prepared agents (C) and, where appropriate, (D) may contain various other ingredients.

The cosmetic ingredients that can be used optionally in the cosmetic carrier may be any suitable ingredients to impart further positive properties to the agent. For example, cosmetic ingredients from the group of thickening or film-forming polymers, surface-active compounds from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

Pigment and/or a Direct Dye in the Agent (C)

In the course of the work leading to the present disclosure, it was observed that the films formed on the keratin material possessed not only good rub fastness but also particularly high color intensity when a coloring compound from the group of pigments and/or direct dyes was used in the process. The use of pigments has proved to be particularly preferable.

The colorant compound(s) may be selected from the group of pigments and direct dyes, where direct dyes may also be photochromic dyes and thermochromic dyes.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A beaker glass is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, a multicomponent packaging unit (kit-of-parts) according to the present disclosure is exemplified in that the agent (C) contains at least one color-imparting compound from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred colorants from the group of pigments according to the present disclosure. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, muscovite or phlogopite, is coated with a metal oxide.

In a very particularly preferred embodiment, a multi-component packaging unit (kit-of-parts) according to the present disclosure is exemplified in that the agent (C) contains at least one coloring compound from the group of inorganic pigments, which is selected from the group of colored metal oxides, metal hydroxides, metal oxide hydrates, silicates, metal sulfides, complex metal cyanides, metal sulfates, bronze pigments and/or colored pigments based on mica or mica coated with at least one metal oxide and/or a metal oxychloride.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, a multicomponent packaging unit (kit-of-parts) according to the present disclosure is exemplified in that the agent (C) comprises at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a multicomponent packaging unit (kit-of-parts) according to the present disclosure is exemplified in that the agent (C) comprises at least one coloring compound selected from mica- or mica-based pigments reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck®, Ariabel® and Unipure® from Sensient®, Prestige® from Eckart® Cosmetic Colors and Sunshine® from Sunstar®.

Particularly preferred color pigments with the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491(Iron oxides), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES).

Other particularly preferred color pigments with the trade name Xirona® are for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name Unipure® are for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica.
Timiron® Synwhite Satin, Merck, Synthetic Fluorphlogopite, Titanium Dioxide, Tin Oxide
Timiron® Super Blue, Merck, Mica, CI 77891 (Titanium Dioxide)
Timiron® Diamond Cluster MP 149, Merck, Mica, CI 77891 (Titanium Dioxide)
Timiron® Splendid Gold, Merck, CI 77891 (titanium dioxide), mica, silicon dioxide
Timiron® Super Sulver, Merck, Mica, CI 77891 (Titanium Dioxide).

In a further embodiment, the agent (C) used in the kit-of-parts according to the present disclosure may also contain one or more colorant compounds from the group of organic pigments.

The organic pigments according to the present disclosure are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindigo, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly preferred embodiment, a multicomponent packaging unit (kit-of-parts) according to the present disclosure is exemplified in that the agent (C) contains at least one coloring compound from the group of organic pigments selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lacquer. In the sense of the present disclosure, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the pigments according to the present disclosure is particularly preferred. It is also preferred if the pigments used have a certain particle size. This particle size leads on the one hand to an even distribution of the pigments in the formed polymer film and on the other hand avoids a rough hair or skin feeling after application of the cosmetic product. According to the present disclosure, it is therefore advantageous if the at least one pigment has an average particle size D₅₀ of about 1.0 to about 50 μm, preferably about 5.0 to about 45 μm, preferably about 10 to about 40 μm, about 14 to about 30 μm. The average particle size D₅₀, for example, can be determined using dynamic light scattering (DLS).

Pigments with a specific shaping may also have been used to color the keratin material. For example, a pigment based on a lamellar and/or a lenticular substrate platelet can be used. Furthermore, coloring based on a substrate platelet comprising a vacuum metallized pigment is also possible.

In a further embodiment, a process according to the present disclosure may be exemplified in that the corresponding agent also comprises one or more colorant compounds selected from the group of lamellar substrate platelet-based pigments, lenticular substrate platelet-based pigments and vacuum metallized pigments.

The substrate platelets of this type have an average thickness of at most about 50 nm, preferably less than about 30 nm, particularly preferably at most about 25 nm, for example at most about 20 nm. The average thickness of the substrate platelets is at least about 1 nm, preferably at least about 2.5 nm, particularly preferably at least about 5 nm, for example at least about 10 nm. Preferred ranges for substrate platelet thickness are about 2.5 to about 50 nm, about 5 to about 50 nm, about 10 to about 50 nm; about 2.5 to about 30 nm, about 5 to about 30 nm, about 10 to about 30 nm; about 2.5 to about 25 nm, about 5 to about 25 nm, about 10 to about 25 nm, about 2.5 to about 20 nm, about 5 to about 20 nm, and about 10 to about 20 nm. Preferably, each substrate plate has a thickness that is as uniform as possible.

Due to the low thickness of the substrate platelets, the pigment exhibits particularly high hiding power.

The substrate plates have a monolithic structure. Monolithic in this context means having a single closed unit without fractures, stratifications, or inclusions, although structural changes may occur within the substrate platelets. The substrate platelets are preferably homogeneously structured, i.e., there is no concentration gradient within the platelets. In particular, the substrate platelets do not have a layered structure and do not have any particles or particles distributed in them.

The size of the substrate platelet can be adjusted to the respective application purpose, especially the desired effect on the keratinic material. Typically, the substrate platelets have an average largest diameter of about 2 to about 200 μm, especially about 5 to about 100 μm.

In a preferred design, the aspect ratio, expressed by the ratio of the average size to the average thickness, is at least about 80, preferably at least about 200, more preferably at least about 500, more preferably more than about 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for about 3 minutes.

The substrate platelets can be composed of any material that can be formed into platelet shape.

They can be of natural origin, but also synthetically produced. Materials from which the substrate platelets can be constructed include metals and metal alloys, metal oxides, preferably aluminum oxide, inorganic compounds, and minerals such as mica and (semi-)precious stones, and plastics. Preferably, the substrate platelets are constructed of metal (alloy).

Any metal suitable for metallic luster pigments can be used. Such metals include iron and steel, as well as all air and water resistant (semi)metals such as platinum, zinc, chromium, molybdenum and silicon, and their alloys such as aluminum bronzes and brass. Preferred metals are aluminum, copper, silver, and gold. Preferred substrate platelets include aluminum platelets and brass platelets, with aluminum substrate platelets being particularly preferred.

Lamellar substrate platelets are exemplified by an irregularly structured edge and are also referred to as “cornflakes” due to their appearance.

Due to their irregular structure, pigments based on lamellar substrate platelets generate a high proportion of scattered light. In addition, pigments based on lamellar substrate platelets do not completely cover the existing color of a keratinous material, and effects analogous to natural graying can be achieved, for example.

Lenticular (=lens-shaped) substrate platelets have a regular round edge and are also called “silver dollars” due to their appearance. Due to their regular structure, the proportion of reflected light predominates in pigments based on lenticular substrate platelets.

Vacuum metallized pigments (VMP) can be obtained, for example, by releasing metals, metal alloys or metal oxides from suitably coated films. They are exemplified by a particularly low thickness of the substrate platelets in the range of about 5 to about 50 nm and a particularly smooth surface with increased reflectivity. Substrate platelets comprising a vacuum metallized pigment are also referred to as VMP substrate platelets in the context of this application. VMP substrate platelets of aluminum can be obtained, for example, by releasing aluminum from metallized films.

The metal or metal alloy substrate plates can be passivated, for example by anodizing (oxide layer) or chromating.

Uncoated lamellar, lenticular and/or VPM substrate plates, especially those made of metal or metal alloy, reflect the incident light to a high degree and create a light-dark flop but no color impression.

A color impression can be created by optical interference effects, for example. Such pigments can be based on at least single-coated substrate platelets. These show interference effects by superimposing differently refracted and reflected light beams.

Accordingly, preferred pigments, pigments based on a coated lamellar substrate platelet. The substrate platelet preferably has at least one coating B of a highly refractive metal oxide having a coating thickness of at least about 50 nm. There is preferably another coating A between the coating B and the surface of the substrate platelet. If necessary, there is a further coating C on the layer B, which is different from the layer B underneath.

Suitable materials for coatings A, B and C are all substances that can be applied to the substrate platelets in a film-like and permanent manner and, in the case of layer A and B, have the required optical properties. Coating part of the surface of the substrate platelets is sufficient to obtain a pigment with a glossy effect. For example, only the top and/or bottom of the substrate platelets may be coated, with the side surface(s) omitted. Preferably, the entire surface of the optionally passivated substrate platelets, including the side surfaces, is covered by coating B. The substrate platelets are thus completely enveloped by coating B. This improves the optical properties of the pigment and increases its mechanical and chemical resistance. The above also applies to layer A and preferably also to layer C if present.

Although multiple coatings A, B and/or C may be present in each case, the coated substrate platelets preferably have only one coating A, B and, if present, C in each case.

The coating B is composed of at least one highly refractive metal oxide. Highly refractive materials have a refractive index of at least about 1.9, preferably at least about 2.0, and more preferably at least about 2.4. Preferably, the coating B comprises at least about 95 wt. %, more preferably at least about 99 wt. %, of high refractive index metal oxide(s).

The coating B has a thickness of at least about 50 nm. Preferably, the thickness of coating B is no more than about 400 nm, more preferably no more than about 300 nm.

Highly refractive metal oxides suitable for coating B are preferably selectively light-absorbing (i.e. colored) metal oxides, such as iron(III) oxide (α- and γ-Fe2O3, red), cobalt(II) oxide (blue), chromium(III) oxide (green), titanium(III) oxide (blue, usually present in admixture with titanium oxynitrides and titanium nitrides), and vanadium(V) oxide (orange), and mixtures thereof. Colorless high-index oxides such as titanium dioxide and/or zirconium oxide are also suitable.

Coating B may contain a selectively absorbing dye, preferably about 0.001 to about 5 wt. %, particularly preferably about 0.01 to about 1 wt. %, in each case based on the total amount of coating B. Suitable dyes are organic and inorganic dyes which can be stably incorporated into a metal oxide coating.

The coating A preferably has at least one low refractive index metal oxide and/or metal oxide hydrate. Preferably, coating A comprises at least about 95 wt. %, more preferably at least about 99 wt. %, of low refractive index metal oxide (hydrate). Low refractive index materials have a refractive index of about 1.8 or less, preferably about 1.6 or less.

Low refractive index metal oxides suitable for coating A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. The coating A preferably has a thickness of about 1 to about 100 nm, particularly preferably about 5 to about 50 nm, especially preferably about 5 to about 20 nm.

Preferably, the distance between the surface of the substrate platelets and the inner surface of coating B is at most about 100 nm, particularly preferably at most about 50 nm, especially preferably at most about 20 nm. By ensuring that the thickness of coating A, and thus the distance between the surface of the substrate platelets and coating B, is within the range specified above, it is possible to ensure that the pigments have a high hiding power.

If the pigment based on a lamellar substrate platelet has only one layer A, it is preferred that the pigment has a lamellar substrate platelet of aluminum and a layer A of silica. If the pigment based on a lamellar substrate platelet has a layer A and a layer B, it is preferred that the pigment has a lamellar substrate platelet of aluminum, a layer A of silica and a layer B of iron oxide.

According to a preferred embodiment, the pigments have a further coating C of a metal oxide (hydrate), which is different from the underlying coating B. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

The coating C preferably has a thickness of about 10 to about 500 nm, more preferably about 50 to about 300 nm. By providing coating C, for example based on TiO₂, better interference can be achieved while maintaining high hiding power.

Layers A and C serve as corrosion protection as well as chemical and physical stabilization. Particularly preferred layers A and C are silica or alumina applied by the sol-gel process. This method comprises dispersing the uncoated lamellar substrate flakes or the lamellar substrate flakes already coated with layer A and/or layer B in a solution of a metal alkoxide such as tetraethyl orthosilicate or aluminum triisopropoxide (usually in a solution of organic solvent or a mixture of organic solvent and water with at least about 50% by weight organic solvent such as a C1 to C4 alcohol), and adding a weak base or acid to hydrolyze the metal alkoxide, thereby forming a film of the metal oxide on the surface of the (coated) substrate flakes.

Layer B can be produced, for example, by hydrolytic decomposition of one or more organic metal compounds and/or by precipitation of one or more dissolved metal salts, as well as any subsequent post-treatment (for example, transfer of a formed hydroxide-containing layer to the oxide layers by annealing).

Although each of the coatings A, B and/or C may be composed of a mixture of two or more metal oxide(hydrate)s, each of the coatings is preferably composed of one metal oxide(hydrate).

The pigments based on coated lamellar or lenticular substrate platelets, or the pigments based on coated VMP substrate platelets preferably have a thickness of about 70 to about 500 nm, particularly preferably about 100 to about 400 nm, especially preferably about 150 to about 320 nm, for example about 180 to about 290 nm. Due to the low thickness of the substrate platelets, the pigment exhibits particularly high hiding power. The low thickness of the coated substrate platelets is achieved by keeping the thickness of the uncoated substrate platelets low, but also by adjusting the thicknesses of the coatings A and, if present, C to as small a value as possible. The thickness of coating B determines the color impression of the pigment.

The adhesion and abrasion resistance of pigments based on coated substrate platelets in keratinic material can be significantly increased by additionally modifying the outermost layer, layer A, B or C depending on the structure, with organic compounds such as silanes, phosphoric acid esters, titanates, borates, or carboxylic acids. In this case, the organic compounds are bonded to the surface of the outermost, preferably metal oxide-containing, layer A, B, or C. The outermost layer denotes the layer that is spatially farthest from the lamellar substrate platelet. The organic compounds are preferably functional silane compounds that can bind to the metal oxide-containing layer A, B, or C. These can be either mono- or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3-methacryloxy-propyltriethoxysilane, 3-acryloxypropyltrimethoxysilane, 2-methacryloxyethyl-triethoxysilane, 2-acryloxyethyltriethoxysilane, 3-methacryloxypropyltris(methoxyethoxy)silane, 3-methacryloxypropyltris(butoxyethoxy)silane, 3-methacryloxy-propyltris(propoxy)silane, 3-methacryloxypropyltris(butoxy)silane, 3-acryloxy-propyltris(methoxyethoxy)silane, 3-acryloxypropyltris(butoxyethoxy)silane, 3-acryl-oxypropyltris(butoxy)silane, vinyltrimethoxysilane, vinyltriethoxysilane, vinylethyl dichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, an alkylsilane or arylsilane, can be conducted. This has only one functional group, which can covalently bond to the surface pigment based on coated lamellar substrate platelets (i.e., to the outermost metal oxide-containing layer) or, if not completely covered, to the metal surface. The hydrocarbon residue of the silane points away from the pigment. Depending on the type and nature of the hydrocarbon residue of the silane, a varying degree of hydrophobicity of the pigment is achieved. Examples of such silanes include hexadecyltrimethoxysilane, propyltrimethoxysilane, etc. Particularly preferred are pigments based on silica-coated aluminum substrate platelets surface-modified with a monofunctional silane. Octyltrimethoxysilane, octyltriethoxysilane, hecadecyltrimethoxysilane and hecadecyltriethoxysilane are particularly preferred. Due to the changed surface properties/hydrophobization, an improvement can be achieved in terms of adhesion, abrasion resistance and alignment in the application.

Suitable pigments based on a lamellar substrate platelet include, for example, the pigments of the VISIONAIRE® series from Eckart®.

Pigments based on a lenticular substrate platelet are available, for example, under the name Alegrace® Gorgeous from the company Schlenk® Metallic Pigments GmbH.

Pigments based on a substrate platelet comprising a vacuum metallized pigment are available, for example, under the name Alegrace® Marvelous or Alegrace® Aurous from the company Schlenk® Metallic Pigments GmbH.

In a further embodiment, a multicomponent packaging unit (kit-of-parts) according to the present disclosure is exemplified in that the agent (C)—based on the total weight of the agent (C)—contains one or more pigments in a total amount of from about 0.001 to about 20 wt. %, from about 0.05 to about 5 wt. %.

As coloring compounds, the compositions according to the present disclosure may also contain one or more direct dyes. Direct dyes are dyes that are applied directly to the hair and do not require an oxidative process to develop the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments. Preferably, the direct dyes within the meaning of the present disclosure have a solubility in water (760 mmHg) at 25° C. of more than 1.0 g/L. In particular, the direct dyes within the meaning of the present disclosure have a solubility in water (760 mmHg) at 25° C. of more than 1.5 g/L.

Direct dyes can be divided into anionic, cationic, and nonionic direct dyes.

In a further preferred embodiment, a multicomponent packaging unit (kit-of-parts) according to the present disclosure is exemplified in that the agent (C) comprises at least one anionic, cationic and/or nonionic direct dye.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51, and Basic Red 76.

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—OO⁻, —SO₃— present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential feature of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF®), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Real red D, FD&C Red Nr. 2, Food Red 9, Naphthol red S), Acid Red 33 (Red 33, Fuchsia Red, D&C Red 33, CI 17200), Acid Red 35 (CI C.I. 18065), Acid Red 51 (CI 45430, Pyrosin B, Tetraiodofluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido Blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. An agitator is added. Then add 100 ml of water. This mixture is heated to 25° C. on a magnetic stirrer while stirring. It is stirred for 60 minutes. The aqueous mixture is then visually assessed. If there are still undissolved radicals, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).
Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is readily soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a very high-water solubility of more than 20 wt. %.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl} {4-[(N-ethyl(3-sulfonatobenzyl)imino]-2,5-cyclohexadien-1-ylidene}methyl)-benzenesulfonate and has a water solubility of greater than 20 wt. % (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to change its color depending reversibly or irreversibly on irradiation with light, especially UV light. This can be done by changing both the intensity and/or the wavelength maximum.

The colorant compound(s) are preferably used in certain ranges of amounts in the agent (C). Preferably, the agent (C) contains—based on the total weight of the agent (C) —one or more colorant compounds in a total amount of from about 0.01 to about 20.0 wt. %, preferably from about 0.1 to about 15.0 wt. %, further preferably from about 0.2 to about 10.0 wt. % and most preferably from about 0.3 to about 5.0 wt. %.

### Film-Forming Polymers in the Agent (D)

Optionally, the multicomponent packaging unit according to the present disclosure may comprise a further separately prepared agent (D) comprising at least one film-forming polymer.

Polymers are macromolecules with a molecular weight of at least about 1000 g/mol, preferably of at least about 2500 g/mol, particularly preferably of at least about 5000 g/mol, which have identical, repeating organic units. The polymers of the present disclosure may be synthetically produced polymers which are manufactured by polymerization of one type of monomer or by polymerization of several types of monomer which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. For the purposes of the present disclosure, it is preferred that the maximum molecular weight of the film-forming hydrophobic polymer (c) is not more than about 107 g/mol, preferably not more than about 106 g/mol and particularly preferably not more than about 105 g/mol.

In the sense of the present disclosure, a film-forming polymer is a polymer which can form a film on a substrate, for example on a keratinic material or a keratinic fiber. The formation of a film can be demonstrated, for example, by looking at the keratin material treated with the polymer under a microscope.

The film-forming polymers can be hydrophilic or hydrophobic.

In a first embodiment, it may be preferred to use at least one hydrophobic film-forming polymer in the agent (D).

A hydrophobic polymer is a polymer that has a solubility in water at 25° C. (760 mmHg) of less than 1 wt. %.

The water solubility of the film-forming, hydrophobic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1 wt. %.

These include acrylic acid-type polymers, polyurethanes, polyesters, polyamides, polyureas, cellulose polymers, nitrocellulose polymers, silicone polymers, acrylamide-type polymers, and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further preferred embodiment, an agent (D) according to the present disclosure is exemplified in that it comprises at least one film-forming, hydrophobic polymer selected from the group of the copolymers of acrylic acid, the copolymers of methacrylic acid, the homopolymers or copolymers of acrylic acid esters, the homopolymers or copolymers of methacrylic acid esters homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

The film-forming hydrophobic polymers, which are selected from the group of synthetic polymers, polymers obtainable by radical polymerization or natural polymers, have proved to be particularly suitable for solving the problem according to the present disclosure.

Other particularly well-suited film-forming hydrophobic polymers can be selected from the homopolymers or copolymers of olefins, such as cycloolefins, butadiene, isoprene or styrene, vinyl ethers, vinyl amides, the esters, or amides of (meth)acrylic acid having at least one C₁-C₂₀ alkyl group, an aryl group or a C₂-C₁₀ hydroxyalkyl group.

Other film-forming hydrophobic polymers may be selected from the homo- or copolymers of isooctyl (meth)acrylate; isonononyl (meth)acrylate; 2-ethylhexyl (meth)acrylate; lauryl (meth)acrylate; isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate and/or mixtures thereof.

Other film-forming hydrophobic polymers may be selected from the homo- or copolymers of (meth)acrylamide; N-alkyl-(meth)acrylamides, in those with C2-C18 alkyl groups, such as N-ethyl-acrylamide, N-tert-butyl-acrylamide, le N-octyl-acrylamide; N-di(C1-C4)alkyl-(meth)acrylamide.

Other preferred anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn® 33 from Rohm® & Haas. Copolymers of acrylic acid, methacrylic acid or their C₁-C₆ alkyl esters and the esters of an ethylenically unsaturated acid and an alkoxylated fatty alcohol are also preferred. Suitable ethylenically unsaturated acids are especially acrylic acid, methacrylic acid and itaconic acid; suitable alkoxylated fatty alcohols are especially steareth-20 or ceteth-20.

Very particularly preferred polymers on the market are, for example, Aculyn® 22 (Acrylates/Steareth-20 Methacrylate Copolymer), Aculyn® 28 (Acrylates/Beheneth-25 Methacrylate Copolymer), Structure 2001® (Acrylates/Steareth-20 Itaconate Copolymer), Structure 3001® (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or the Rohm® und Haas distributed Soltex® OPT (Acrylates/C12-22 Alkyl methacrylate Copolymer).

The homo- and copolymers of N-vinylpyrrolidone, vinylcaprolactam, vinyl-(C1-C6)alkyl-pyrrole, vinyl-oxazole, vinyl-thiazole, vinylpyrimidine, vinylimidazole can be named as suitable polymers based on vinyl monomers.

Furthermore, the copolymers octylacrylamide/acrylates/butylaminoethyl-methacrylate copolymer, as commercially marketed under the trade names AMPHOMER® or LOVOCRYL® 47 by NATIONAL STARCH, or the copolymers of acrylates/octylacrylamides marketed under the trade names DERMACRYL® LT and DERMACRYL® 79 by NATIONAL STARCH are particularly suitable.

Suitable olefin-based polymers include homopolymers and copolymers of ethylene, propylene, butene, isoprene and butadiene.

In another embodiment, the film-forming hydrophobic polymers may be the block copolymers comprising at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF® under the trade name “Luvitol HSB”.

Good results were also obtained when the agent (D) contained at least one film-forming polymer selected from the group of homopolymers and copolymers of acrylic acid, homopolymers and copolymers of methacrylic acid, homopolymers and copolymers of acrylic esters, homopolymers and copolymers of methacrylic esters, homopolymers and copolymers of acrylic amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a further embodiment, it may be preferred to use, in the agent (D), at least one hydrophilic film-forming polymer.

By a hydrophilic polymer is meant a polymer that has a solubility in water at 25° C. (760 mmHg) of more than about 1 wt. %, preferably more than about 2 wt. %.

The water solubility of the film-forming, hydrophilic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1 wt. %.

Nonionic, anionic, and cationic polymers can be used as film-forming, hydrophilic polymers.

Suitable film-forming hydrophilic polymers can be selected, for example, from the group of polyvinylpyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides and/or acrylamide (co)polymers.

Furthermore, it is particularly preferred to use polyvinylpyrrolidone (PVP) and/or a vinylpyrrolidone-containing copolymer as film-forming hydrophilic polymer.

In another very particularly preferred embodiment, an agent (D) according to the present disclosure is exemplified in that it comprises at least one film-forming hydrophilic polymer selected from the group of polyvinylpyrrolidone (PVP) and the copolymers of polyvinylpyrrolidone.

It is further preferred if the agent (D) according to the present disclosure contains polyvinylpyrrolidone (PVP) as the film-forming hydrophilic polymer. Particularly well-suited polyvinylpyrrolidones are available, for example, under the name Luviskol® K from BASF® SE, especially Luviskol® K 90 or Luviskol® K 85 from BASF® SE.

The polymer PVP K30, which is marketed by Ashland® (ISP, POI Chemical), can also be used as another explicitly very well suited polyvinylpyrrolidone (PVP). PVP K 30 is a polyvinylpyrrolidone which is highly soluble in cold water and has the CAS number 9003-39-8. The molecular weight of PVP K 30 is about 40000 g/mol.

Other particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF®.

The use of film-forming hydrophilic polymers from the group of copolymers of polyvinylpyrrolidone has also led to particularly good and washfast color results.

Vinylpyrrolidone-vinyl ester copolymers, such as those marketed under the trademark Luviskol® (BASF®), are particularly suitable film-forming hydrophilic polymers. Luviskol® VA 64 and Luviskol® VA 73, both vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly preferred in cosmetic compositions.

Vinylpyrrolidone-vinyl acetate copolymers are marketed under the name Luviskol® VA by BASF® SE. For example, a VP/Vinyl Caprolactam/DMAPA Acrylates copolymer is sold under the trade name Aquaflex® SF-40 by Ashland® Inc. For example, a VP/DMAPA acrylates copolymer is marketed by Ashland® under the name Styleze® CC-10 and is a highly preferred vinylpyrrolidone-containing copolymer.

Other suitable copolymers of polyvinylpyrrolidone may also be those obtained by reacting N-vinylpyrrolidone with at least one further monomer from the group of V-vinylformamide, vinyl acetate, ethylene, propylene, acrylamide, vinylcaprolactam, vinylcaprolactone and/or vinyl alcohol.

In another very particularly preferred embodiment, an agent (D) according to the present disclosure is exemplified in that it comprises at least one film-forming hydrophilic polymer selected from the group of polyvinylpyrrolidone (PVP), vinylpyrrolidone/vinyl acetate copolymers, vinylpyrrolidone/styrene copolymers, vinylpyrrolidone/ethylene copolymers, vinylpyrrolidone/propylene copolymers, vinylpyrrolidone/vinylcaprolactam copolymers, vinylpyrrolidone/vinylformamide copolymers and/or vinylpyrrolidone/vinyl alcohol copolymers.

Another suitable copolymer of vinylpyrrolidone is the polymer known under the INCI designation maltodextrin/VP copolymer.

Furthermore, intensively dyed keratin material, especially hair, with particularly good wash fastness could be obtained if a non-ionic, film-forming, hydrophilic polymer was used as the film-forming, hydrophilic polymer.

In the context of a further embodiment, it may be preferred if the agent (D), comprises at least one non-ionic, film-forming, hydrophilic polymer.

According to the present disclosure, a non-ionic polymer is understood to be a polymer which in a protic solvent—such as water—under standard conditions does not carry structural units with permanent cationic or anionic groups, which must be compensated by counterions while maintaining electron neutrality. Cationic groups include quaternized ammonium groups but not protonated amines. Anionic groups include carboxylic and sulphonic acid groups.

Preference is given to products containing, as a non-ionic, film-forming, hydrophilic polymer, at least one polymer selected from the group of
Polyvinylpyrrolidone,
Copolymers of N-vinylpyrrolidone and vinyl esters of carboxylic acids having 2 to about 18 carbon atoms of N-vinylpyrrolidone and vinyl acetate,
Copolymers of N-vinylpyrrolidone and N-vinylimidazole and methacrylamide,
Copolymers of N-vinylpyrrolidone and N-vinylimidazole and acrylamide,
Copolymers of N-vinylpyrrolidone with N,N-di(C1 to C4)-alkylamino-(C2 to C4)-alkylacrylamide,

If copolymers of N-vinylpyrrolidone and vinyl acetate are used, it is again preferable if the molar ratio of the structural units contained in the monomer N-vinylpyrrolidone to the structural units of the polymer contained in the monomer vinyl acetate is in the range from about 20:80 to about 80:20, in particular from about 30:70 to about 60:40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF® SE.

Another particularly preferred polymer is selected from the INCI designation VP/Methacrylamide/Vinyl Imidazole Copolymer, which is available under the trade name Luviset® Clear from BASF® SE.

Another particularly preferred non-ionic, film-forming, hydrophilic polymer is a copolymer of N-vinylpyrrolidone and N,N-dimethylaminiopropylmethacrylamide, which is sold under the INCI designation VP/DMAPA Acrylates Copolymer e.g., under the trade name Styleze® CC 10 by ISP.

A cationic polymer of interest is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI designation): Polyquaternium-69), which is marketed, for example, under the trade name AquaStyle® 300 (28-32 wt. % active substance in ethanol-water mixture, molecular weight 350000) by ISP.

Other suitable film-forming, hydrophilic polymers include Vinylpyrrolidone-vinylimidazolium methochloride copolymers, as offered under the designations Luviquat© FC 370, FC 550, and the INCI designation Polyquaternium-16 as well as FC 905 and HM 552,
Vinylpyrrolidone-vinylcaprolactam-acrylate terpolymers, as they are commercially available with acrylic acid esters and acrylic acid amides as a third monomer component, for example under the name Aquaflex® SF 40.

Polyquaternium-11 is the reaction product of diethyl sulphate with a copolymer of vinyl pyrrolidone and dimethylaminoethyl methacrylate. Suitable commercial products are available under the names Dehyquart® CC 11 and Luviquat® PQ 11 PN from BASF® SE or Gafquat® 440, Gafquat® 734, Gafquat® 755 or Gafquat® 755N from Ashland® Inc.

Polyquaternium-46 is the reaction product of vinylcaprolactam and vinylpyrrolidone with methylvinylimidazolium methosulfate and is available for example under the name Luviquat® Hold from BASF® SE. Polyquaternium-46 is preferably used in an amount of 1 to 5 wt. %—based on the total weight of the cosmetic composition. It particularly prefers to use polyquaternium-46 in combination with a cationic guar compound. It is even highly preferred that polyquaternium-46 is used in combination with a cationic guar compound and polyquaternium-11.

Suitable anionic film-forming, hydrophilic polymers can be, for example, acrylic acid polymers, which can be in non-crosslinked or crosslinked form. Such products are sold commercially under the trade names Carbopol® 980, 981, 954, 2984 and 5984 by Lubrizol or under the names Synthalen® M and Synthalen® K by 3V Sigma (The Sun Chemicals®, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the group of acrylamides are, for example, polymers which are produced from monomers of (meth)acrylamido-C1-C4-alkyl sulphonic acid or the salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Preferred polymers of the poly(meth)arylamido-C1-C4-alkyl sulphonic acids are cross-linked and at least about 90% neutralized. These polymers can or cannot be cross-linked.

Cross-linked and fully or partially neutralized polymers of the poly-2-acrylamido-2-methylpropane sulfonic acid type are available under the INCI designation “Ammonium Polyacrylamido-2-methyl-propanesulphonates” or “Ammonium Polyacryldimethyltauramides”.

Another preferred polymer of this type is the cross-linked poly-2-acrylamido-2-methyl-propanesulphonic acid polymer marketed by Clamant under the trade name Hostacerin® AMPS, which is partially neutralized with ammonia.

In a further embodiment, it may be preferred if the agent (D) comprises at least one anionic, film-forming, polymer.

In this context, the best results were obtained when the agent (D) contains at least one film-forming polymer comprising at least one structural unit of formula (P-I) and at least one structural unit of formula (P-II)

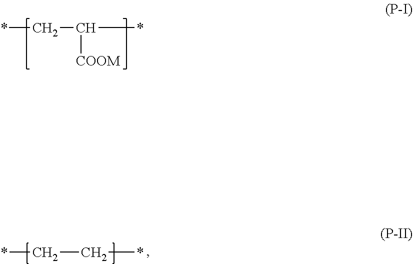

where
M is a hydrogen atom or ammonium (NH₄), sodium, potassium, 12 magnesium or 12 calcium.

When M represents a hydrogen atom, the structural unit of the formula (P-I) is based on an acrylic acid unit.
When M stands for an ammonium counterion, the structural unit of the formula (P-I) is based on the ammonium salt of acrylic acid.
When M stands for a sodium counterion, the structural unit of the formula (P-I) is based on the sodium salt of acrylic acid.
When M stands for a potassium counterion, the structural unit of the formula (P-I) is based on the potassium salt of acrylic acid.
If M stands for a half equivalent of a magnesium counterion, the structural unit of the formula (P-I) is based on the magnesium salt of acrylic acid.
If M stands for a half equivalent of a calcium counterion, the structural unit of the formula (P-I) is based on the calcium salt of acrylic acid.

The film-forming polymer or polymers of the present disclosure are preferably used in certain ranges of amounts in the agent (D). In this context, it has been shown to be preferable if the agent (D)-based on its total weight-contains one or more film-forming polymers in a total amount of from about 0.1 to about 18.0 wt. %, preferably from about 1.0 to about 16.0 wt. %, more preferably from about 5.0 to about 14.5 wt. % and most preferably from about 8.0 to about 12.0 wt. %.

Process for the Treatment of keratinous Material

A further subject of the present application is a process for dyeing keratinous material, in particular human hair, comprising the following steps
(1) Providing a cosmetic composition as already disclosed in detail in the description of the first subject matter of the present disclosure,
(2) Providing an agent (C), wherein the agent (C) comprises at least one pigment and/or a direct dye,
(3) Mixing the composition provided in step (1) with the agent (C) provided in step (2) to produce a ready-to-use colorant,
(4) Application of the ready-to-use colorant prepared in step (3) on the keratinous material,
(5) Exposure of the colorant applied in step (4) to the keratinous material,
(6) Rinse out the dye,
(7) optionally applying an after-treatment agent (D) to the keratinous material, the agent (D) comprising at least one film-forming polymer,
(8) if necessary, exposing the keratinous material to the aftertreatment agent applied in step (7), and
(9) if necessary, rinsing the after-treatment agent (D) out of the keratinous material.

In step (1) of the process, the cosmetic composition according to the present disclosure is provided. This can be done, for example, in the form of a separately prepared blend or concentrate, which is preferably packaged in an airtight manner. Just before application, the user or hairdresser can pick up the agent (C) in step (2) and mix it with the cosmetic composition in step (3).

Mixing in step (3) can be done, for example, by stirring or shaking. Most advantageously, the two preparations are made up separately in two containers, and then, before use, the entire amount of the cosmetic composition provided in step (1) is transferred from its container to the container containing the agent (C).

The cosmetic composition and the agent (C) can be mixed in different proportions.

Particularly preferably, cosmetic composition in step (1) is provided in the form of a highly concentrated, low-water silane blend that is quasi-diluted by mixing with agent (C). For this reason, it is particularly preferred to mix the cosmetic composition provided in step (1) with an excess by weight of agent (C). For example, 1 part by weight of cosmetic composition (siloxane mixture) may be mixed with about 20 parts by weight of agent (C), or 1 part by weight of cosmetic composition (siloxane mixture) may be mixed with 10 parts by weight of agent (C), or 1 part by weight of cosmetic composition (siloxane mixture) may be mixed with about 5 parts by weight of agent (C).

In step (4) of the process, the application-ready agent prepared in step (3) is applied to the keratinous material, to human hair. The application can be done with the help of the gloved hand or with the help of a brush, a spout, or an applicator.

Then, in step (5), the applied agent is allowed to act into or onto the keratinous material. Suitable exposure times here are from about 30 seconds to about 60 minutes, preferably from about 1 to about 30 minutes, further preferably from about 1 to about 20 minutes, and most preferably from about 1 to about 10 minutes.

Then, in step (6), the agent is rinsed off the keratinous material, or hair. Rinsing is preferably done with tap water only.

In steps (7), (8) and (v9), an after-treatment agent (agent (D)) can optionally still be applied to the keratinous material, allowed to act, and then rinsed out again if necessary.

The use of an after-treatment agent (agent (D)) may also be preferred if the process for treating keratinous material is a dyeing process in which a coloring compound, such as in particular in pigment, is still to be applied to the keratinous materials in a downstream step.

Preferably, agents (C) and (D) are the agents that have also been mentioned as preferred or particularly preferred in the description of the kit-of-parts.

Regarding the further preferred embodiments of the multicomponent packaging unit according to the present disclosure and of the process according to the present disclosure, what has been said about the cosmetic composition according to the present disclosure applies mutatis mutandis.

### EXAMPLES

### Preparation of the Agent (A)

In a 500 ml round bottom flask, 23.0 g ethanol (abs.) and 47.90 g methyltriethoxysilane (MTES) were mixed with stirring. This mixture was heated to 50° C. with further stirring. Then 4.404 g of a 1% solution of sulfuric acid in water was added over a period of about 5 minutes. The temperature of the reaction mixture increased to 62° C. and decreased to 55° C. after the addition was completed. Stirring was continued for another 20 minutes. Then 23.95 g of (3-aminopropyl)triethoxysilane (AMEO) was added over a period of about 5 minutes. After completion of the addition, the mixture was stirred at 50° C. for an additional 45 minutes and subsequently poured into an airtight glass jar.
3-Aminopropyltriethoxsilane C₉H₂₃NO₃Si=221.37 g/mol
Methyltriethoxysilane C₇H₁₈O₃Si=178.34 g/mol
23.95 g 3-aminopropyltriethoxsilane (AMEO)=0.1081 mol
3-Aminopropyltriethoxsilane has 3 hydrolysable alkoxy groups per molecule.
47.90 g methyltriethoxysilane (MTES)=0.269 mol
Methyltrimethoxysilane has 3 hydrolysable alkoxy groups per molecule).
4,404 g 1% solution of sulfuric acid in water=4.36 g water
Water H₂O=18.015 g/mol
4.36 g water=0.242 mol

$$X = \frac{[ n ( AMEO ) \times 3 ] + ( n ( MTES ) \times 3 ]}{n ( H 2 O )}$$

$$X = \frac{[ ( 0 , 1081 ) \times 3 ] + [ ( 0 , 269 ) \times 3 ]}{0 , 242} = 4.9$$

### Preparation of the Agent (B)

In a 500 ml round bottom flask, 23.0 g ethanol (abs.) and 46.11 g methyltriethoxysilane (MTES) were mixed with stirring. This mixture was heated to 50° C. with further stirring. Then 8.40 g of a 1% solution of sulfuric acid in water was added over a period of about 5 minutes. The temperature of the reaction mixture increased to 62° C. and decreased to 55° C. after the addition was completed. Stirring was continued for another 20 minutes. Then 23.95 g of (3-aminopropyl)triethoxysilane (AMEO) was added over a period of about 5 minutes. After completion of the addition, the mixture was stirred at 50° C. for an additional 45 minutes and subsequently poured into an airtight glass jar.
3-Aminopropyltriethoxsilane C₉H₂₃NO₃Si=221.37 g/mol
Methyltriethoxysilane C₇H₁₈O₃Si=178.34 g/mol
23.05 g 3-aminopropyltriethoxsilane (AMEO)=0.1081 mol
3-Aminopropyltriethoxsilane has 3 hydrolysable alkoxy groups per molecule.
46.11 g methyltriethoxysilane (MTES)=0.259 mol
Methyltrimethoxysilane has 3 hydrolysable alkoxy groups per molecule).
8.40 g 1% solution of sulfuric acid in water=8.316 g water
Water H₂O=18.015 g/mol
8.316 g water=0.461 mol

$$Y = \frac{[ n ( AMEO ) \times 3 ] + ( n ( MTES ) \times 3 ]}{m ( H 2 O )}$$

$$Y = \frac{[ ( 0 , 1081 ) \times 3 ] + [ ( 0 , 259 ) \times 3 ]}{0 , 461} = 2.4$$

### Preparation of the Cosmetic Composition

For the preparation of the cosmetic composition, 100 g of the agent (A) was mixed with 44 g of the agent (B). The mixture prepared in this way was poured into an airtight glass jar.

### 3. Coloring

For coloring on hair, a ready-to-use coloring agent was first prepared. For this purpose, 10 g of the previously prepared cosmetic composition was mixed with 100 g of the agent (C) (shaking for 3 minutes).

### Preparation (C)

| | | |
|---|---|---|
| Colorona ® Bordeaux, Merck ®, MICA, CI 77491 (IRON | 5.5 | g |
| OXIDES) | | |
| Hydroxyethyl cellulose (Natrosol 250 HR) | 1.0 | g |
| Cetearyl alcohol | 1.5 | g |
| Eumulgin ® B3 (Ceteareth-30) | 1.5 | g |
| Water | Ad 100 | g |

Then one strand of hair (Kerling Euro natural white) was dipped into the ready-to-use dye and left in it for 1 minute. After that, superfluous agent was striped from each strand of hair. Subsequently, each strand of hair was washed with water and dried. Subsequently, the strands were visually evaluated under a daylight lamp.

The strand of hair was dyed with burgundy color in high color intensity.

1. A cosmetic composition for the treatment of keratinous material, comprising a first agent (A) and a second agent (B), wherein the first agent (A) and the second agent (B) are mixed, wherein
the first agent (A) is obtained by reacting one or more organic C₁-C₆ alkoxysilanes with an amount of water, wherein the amount of water is determined according to equation (G-1),
X=[(n_{I}(Alkoxysilane)×n_{II}(Alkoxy groups)]/n(H₂O) (G-1)
where
n(H₂O) is the amount of water used in the agent (A) expressed in moles,
n_{I} is the amount of the organic C₁-C₆ alkoxysilanes used in the agent (A) expressed in moles,
n_{II} is a number of C₁-C₆ alkoxy groups per the organic C₁-C₆ alkoxysilane used in the agent (A), and
X is a number from about 3.0 to about 100, and
the second agent (B) is obtained by reacting one or more organic C₁-C₆ alkoxysilanes with an amount of water, wherein the amount of water is determined according to equation (G-2),
Y=[(nₐ(Alkoxysilane)×n_{b}(Alkoxy groups)]/m(H₂O) (G-2)
where
m(H₂O) is the amount of water used in the agent (B) expressed in moles,
nₐ is the amount of the organic C₁-C₆ alkoxysilanes used in the agent (B) expressed in moles,
nb is a number of C₁-C₆ alkoxy groups per the organic C₁-C₆ alkoxysilane used in the agent (B), and
Y is a number from about 0.1 to about 2.9.

2. The cosmetic composition according to claim 1, wherein the first agent (A) is obtained by reacting the one or more organic C₁-C₆ alkoxysilanes of formula (I) and/or (II) and/or (IV),
R₁R₂N-L-Si(OR₃)ₐ(R₄)_{b} (I)
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a stands for an integer from 1 to 3, and
b stands for the integer 3-a, and
(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),
where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),
(A″″)-Si(R₆″)_{d}″(OR₅″)_{c}″ (III),
wherein
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h radicals are different from 0,
(R₉)ₘSi(OR₁₀)ₖ (IV),
where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

3. The cosmetic composition according to claim 1, wherein the one or more C₁-C₆ organic alkoxysilanes is selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Vinyl trimethoxysilane
Vinyl triethoxysilane
Tetramethoxysilane
Tetraethoxysilane
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane, and
(2-Dimethylaminoethyl)trimethoxysilane.

4. The cosmetic composition according claim 1, wherein
X is equal to a number from about 3.5 to about 85.0.

5. The cosmetic composition according to claim 1, wherein the second agent (B) is obtained by reacting the one or more organic C₁-C₆ alkoxysilanes of formula (I) and/or (II) and/or (IV) with water,
R₁R₂N-L-Si(OR₃)ₐ(R₄)_{b} (I)
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a stands for an integer from 1 to 3, and
b stands for the integer 3-a, and
(R₅O)_{c}(R₆)_{d}Si-(A)ₑ-[NR₇-(A′)]_{f}—[O-(A″)]_{g}—[NR₈-(A′″)]ₕ-Si(R₆′)_{d′}(OR₅′)_{c′} (II),
where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),
(A″″)-Si(R₆″)_{d}″(OR₅″)_{c}″ (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h radicals are different from 0,
(R₉)ₘSi(OR₁₀)ₖ (IV),
where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

6. The cosmetic composition according to claim 1, wherein the one or more C₁-C₆ organic alkoxysilanes is selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Vinyl trimethoxysilane
Vinyl triethoxysilane
Tetramethoxysilane
Tetraethoxysilane
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane, and
(2-Dimethylaminoethyl)trimethoxysilane.

7. The cosmetic composition according to claim 1, wherein
Y is equal to a number from about 0.4 to about 2.8.

8. The cosmetic composition according to claim 1, wherein the cosmetic composition is obtained by mixing the first agent (A) with the second agent (B) in a weight ratio (A)/(B) of from about 1:5 to about 5:1.

9. The cosmetic composition according to claim 1, wherein:
the first agent (A) is obtained by reacting the one or more organic C₁-C₆ alkoxysilanes with the water in the presence of a solvent selected from the group of methanol, ethanol, isopropanol, and a combination thereof, and/or
the second agent (B) is obtained by reacting the one or more organic C₁-C₆ alkoxysilanes with the water in the presence of a solvent selected from the group of methanol, ethanol, isopropanol, and combinations thereof.

10. The cosmetic composition according to claim 1, wherein:
the first agent (A) is obtained by reacting the one or more organic C₁-C₆ alkoxysilanes with the water in the presence of a catalyst selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid 1-hydroxyethane-1,1-diphosphonic acid, sodium hydroxide, potassium hydroxide, magnesium hydroxide, calcium hydroxide, and combinations thereof.

11. A multicomponent packaging unit (kit-of-parts) for dyeing keratinous material, which comprises
a first packaging unit comprising a cosmetic composition comprising a first agent (A) and a second agent (B), wherein the first agent (A) and the second agent (B) are mixed, wherein
the first agent (A) is obtained by reacting one or more organic C₁-C₆ alkoxysilanes with an amount of water, wherein the amount of water is determined according to equation (G-1)
X=[(n_{I}(Alkoxysilane)×n_{II}(Alkoxy groups)]/n(H₂O) (G-1)
where
n(H₂O) is the amount of water used in the agent (A) expressed in moles,
n_{I} is the amount of the organic C₁-C₆ alkoxysilanes used in the agent (A) expressed in moles,
n_{II} is a number of C₁-C₆ alkoxy groups per the organic C₁-C₆ alkoxysilane used in the agent (A), and
X is a number from about 3.0 to about 100, and
the second agent (B) is obtained by reacting one or more organic C₁-C₆ alkoxysilanes with an amount of water, wherein the amount of water is determined according to equation (G-2),
Y=[(nₐ(Alkoxysilane)×n_{b}(Alkoxy groups)]/m(H₂O) (G-2)
where
m(H₂O) is the amount of water used in the agent (B) expressed in moles,
nₐ is the amount of the organic C₁-C₆ alkoxysilanes used in the agent (B) expressed in moles,
nb is a number of C₁-C₆ alkoxy groups per the organic C₁-C₆ alkoxysilane used in the agent (B), and
Y is a number from about 0.1 to about 2.9,
a second packaging unit containing an agent (C), wherein the agent (C) comprises at least one pigment and/or direct dye, and
optionally a third packaging unit containing an agent (D), wherein the agent (D) comprises at least one film-forming polymer.

12. A method for dying keratinous material, the method comprising the steps of:
(1) providing a cosmetic composition comprising a first agent (A) and a second agent (B), wherein the first agent (A) and the second agent (B) are mixed, wherein
the first agent (A) is obtained by reacting one or more organic C₁-C₆ alkoxysilanes with an amount of water, wherein the amount of water is determined according to equation (G-1),
X=[(n_{I}(Alkoxysilane)×n_{II}(Alkoxy groups)]/n(H₂O) (G-1)
where
n(H₂O) is the amount of water used in the agent (A) expressed in moles,
n_{I} is the amount of the organic C₁-C₆ alkoxysilanes used in the agent (A) expressed in moles,
n_{II} is a number of C₁-C₆ alkoxy groups per the organic C₁-C₆ alkoxysilane used in the agent (A), and
X is a number from about 3.0 to about 100, and
the second agent (B) is obtained by reacting one or more organic C₁-C₆ alkoxysilanes with an amount of water, wherein the amount of water is determined according to equation (G-2),
Y=[(nₐ(Alkoxysilane)×n_{b}(Alkoxy groups)]/m(H₂O) (G-2)
where
m(H₂O) is the amount of water used in the agent (B) expressed in moles,
nₐ is the amount of the organic C₁-C₆ alkoxysilanes used in the agent (B) expressed in moles,
nb is a number of C₁-C₆ alkoxy groups per the organic C₁-C₆ alkoxysilane used in the agent (B), and
Y is a number from about 0.1 to about 2.9,
(2) provding an agent (C), wherein the agent (C) comprises at least one pigment and/or a direct dye,
(3) mixing the cosmetic composition provided in step (1) with the agent (C) prepared in step (2) to produce a ready-to-use colorant,
(4) applying the ready-to-use colorant prepared in step (3) on the keratinous material,
(5) exposing the ready-to-use colorant applied in step (4) to the keratinous material, and
(6) rinsing the ready-to-use colorant out of the keratinous material.

13. The method of claim 12, further comprising:
(7) applying an after treatment agent (D) to the keratinous material, wherein the agent (D) comprises a film forming polymer.

14. The method of claim 13, further comprising:
(8) exposing the keratinous material to the after treatment agent (D) applied in step (7).

15. The method of claim 14, further comprising:
Rinsing the after treatment agent (D) out of the keratinous material.

16. The cosmetic composition of claim 1, wherein:
X is a number from about 4.3 to about 5.0.

17. The cosmetic composition of claim 1, wherein:
Y is a number from about 1.7 to about 2.4.

18. The cosmetic composition of claim 1, wherein:
the cosmetic composition is obtained by mixing the first agent (A) with the second agent (B) in a weight ratio (A)/(B) of from about 2:1 to about 3:1.

19. The cosmetic composition of claim 1, wherein:
X is a number from about 3.9 to about 45.0.

20. The cosmetic composition of claim 1, wherein:
Y is a number from about 1.0 to about 2.6.
